(19) United States
(12) Patent Application Publication
(10) Pub. No.: US 2005/0209250 A1
(43) Pub. Date: Sep. 22, 2005

(54) Therapeutic combinations of atypical antipsychotics with corticotropin releasing factor antagonists

(75) Inventor: Steve Romano, New York, NY (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/058,329

(22) Filed: Feb. 14, 2005

Related U.S. Application Data
(60) Provisional application No. 60/544,731, filed on Feb. 13, 2004.

(51) Int. Cl. A61K 31/519; A61K 31/44; A61K 31/416

(52) U.S. Cl. 514/259.3; 514/262.1; 514/352; 514/406

(57) ABSTRACT

The present invention is directed to a pharmaceutical compositions for treating, for example, mood disorders or conditions, psychotic disorders or conditions, or a combination thereof, in a mammal such as a human, the composition comprising (a) an atypical antipsychotic, a prodrug thereof or a pharmaceutically acceptable salt of the atypical antipsychotic or prodrug thereof, (b) a corticotropin releasing factor antagonist, a prodrug thereof, or pharmaceutically acceptable salt of said corticotropin releasing factor antagonist or prodrug thereof, and optionally (c) a pharmaceutically acceptable vehicle, carrier or diluent. The present invention is also directed to a method for treating one or more disorders or conditions described in the previous sentence, the method comprising administering to a mammal in need of such treatment components (a) and (b) described in the previous sentence, wherein (a) and (b) are each optionally and independently administered together with a pharmaceutically acceptable vehicle, carrier or diluent.

### FIELD OF THE INVENTION

The present invention relates to pharmaceutical compositions comprising combinations of an atypical antipsychotic, a prodrug thereof or a pharmaceutically acceptable salt of the atypical antipsychotic or prodrug thereof, and a corticotropin releasing factor antagonist, a prodrug thereof or a pharmaceutically acceptable salt of said corticotropin releasing factor antagonist or prodrug thereof, kits containing such combinations and methods of using such combinations to treat mammals, including humans, suffering from treatment-resistant anxiety disorders, psychotic disorders or conditions, mood disorders or conditions, or a combination thereof. This invention also relates to additive and synergistic combinations of atypical antipsychotic, a prodrug thereof or a pharmaceutically acceptable salt of the atypical antipsychotic or prodrug thereof, and a corticotropin releasing factor antagonist, a prodrug thereof or a pharmaceutically acceptable salt of said corticotropin releasing factor antagonist or prodrug thereof, whereby the additive and synergistic combinations are useful in treating mammals, including humans, suffering from treatment-resistant anxiety disorders, psychotic disorders or conditions, mood disorders or conditions, or a combination thereof.

### BACKGROUND OF THE INVENTION

Atypical antipsychotics offer several clinical benefits over the conventional antipsychotics, which were the mainstays of care until the past decade. The principal mechanism underlying the many clinical benefits of the atypical antipsychotics is their ability to separate the antipsychotic effect from the extrapyramidal side effect (EPS). The distinct advantages over traditional antipsychotic medications include greater improvement in negative and cognitive symptoms, better antidepressant and mood stabilization effects, lower risk of parkinsonian side effects and tardive dyskinesia, and greater efficacy in otherwise refractory or treatment-resistant patients.

The differences in clinical profile between atypical and conventional antipsychotics can be understood in terms of their different pharmacological profiles. The conventional antipsychotics are antagonists of dopamine (D₂) receptors. The atypical antipsychotics also have D₂ antagonistic properties, but possess different binding kinetics to these receptors and activity at other receptors, particularly 5-HT_{2A}, 5-HT_{2c} and 5-HT_{1D} (Schmidt B et al, Soc. Neurosci. Abstr. 24:2177, 1998). For example, an atypical antipsychotic may have dual antagonism of serotonin 5-HT_{2A} and dopamine D₂.

Examples of atypical antipsychotics for use in the present invention are the compounds generically and specifically disclosed in U.S. Pat. No. 4,831,301, particularly ziprasidone (Geodon®), U.S. Pat. No. 5,229,382, particularly olanzapine (Zyprexa®), U.S. Pat. No. 3,539,573, particularly clozapine (Clozaril®), U.S. Pat. No. 4,804,663, particularly risperidone (Risperdal®), U.S. Pat. No. 4,710,500, particularly sertindole, U.S. Pat. No. 4,879,288, particularly quetiapine (Seroquel®), U.S. Pat. No. 4,734,416, particularly aripiprazole (Abilify®), and U.S. Pat. No. 4,401,822, particularly amisulpride, or pharmaceutically acceptable salts thereof.

Commonly assigned U.S. Pat. Nos. 4,831,031, 4,883,795, 5,229,382, and 6,245,766, which are hereby incorporated by reference, each disclose that ziprasidone has utility in the treatment of treatment-resistant anxiety disorders, psychotic disorders, and mood disorders.

Psychotic disorders or conditions, such as schizoaffective disorder, are serious mental disorders characterized by loss of contact with reality (psychosis), hallucinations (false perceptions), delusions (false beliefs), abnormal thinking, flattened affect, diminished motivation, and disturbed work and social functioning.

Mood disorders or conditions, also known as affective disorders, are a group of heterogeneous, typically recurrent illnesses including unipolar (depressive) and bipolar (manic-depressive) disorders, dysthymic disorder, and cyclothymic disorder that are characterized by pervasive mood disturbances, psychomotor dysfunction, and vegetative symptoms. Mood disorders may affect 20% of women and 12% of men during their lifetime. They are the most prevalent of psychiatric disorders, accounting for as many as 65% of psychiatric outpatients, and 10% of all patients seen in nonpsychiatric medical settings (The Merck Manual, 17^{th} ed., Merck & Co. 1999, p. 1526). Lithium, the standard of care for mood disorder, has a response rate of only 50%, and is associated with side effects. Antipsychotic agents are also clinically used in this patient population.

Simplification of the regimen for the treatment of mood disorders or conditions, such as psychotic depression, or of psychotic disorders or conditions, such as schizoaffective disorders, may be achieved by combining two therapeutic agents. The combined treatment reduces the opportunity for patient noncompliance and occurs with a more rigorous schedule. Accordingly, there is a need for pharmaceutical combinations and pharmaceutical kits which employ atypical antipsychotics and another therapeutic agent efficacious for the treatment of conditions such as mood disorders or conditions, psychotic disorders or conditions, or a combination thereof.

Corticotropin releasing factor (CRF) antagonists are another class of therapeutic agents that have been described as effective in the treatment of certain disorders or conditions. CRF antagonists are disclosed in U.S. Pat. Nos. 4,605,642 and 5,063,245. Other CRF antagonists are disclosed in International patent publications WO 95/33750; WO 95/34563; WO 94/13661; WO 94/13644; WO 94/13643; WO 94/13676; WO 94/13677; WO 95/33727; WO 98/05661; WO 98/08847; WO 98/08846; and European patent publications EP 778277 and EP 773023. Yet other CRF antagonists are disclosed in the following patent publications: EP 576350; EP 659747; EP 812831; WO 95/10506; WO 96/35689; WO 96/39400; WO 97/00868; WO 97/14684; WO 97/29109; WO 97/29110; WO 97/35539; WO 97/35580; WO 97/35846; WO 97/44038; WO 97/45421; WO 98/03510; WO 98/08821; WO 98/11075; WO 98/15543; WO 98/21200; WO 98/27066; WO 98/29397; WO 98/29413; WO 98/42699; WO 98/35967; WO 98/42706; WO 98/45295; WO 98/47874; WO 98/47903; WO 98/51312; WO 99/01454; WO 99/01439; WO 99/10350; WO 99/12908; WO 99/00373; WO 99/38868; WO 99/51597; WO 99/51599; WO 99/40089; WO 99/51598; and WO 99/51600. Still more CRF antagonists are disclosed in U.S. Pat. Nos. 5,109,111; 5,132,111; 5,245,009; 5,464,847; 5,493,006; 5,510,458; 5,644,057; 5,663,292; 5,668,145; 5,705,646; 5,712,303; and 5,723,608. An overview of the patent literature on CRF antagonists is provided in T. E. Christos and A. Arvanitis, Exp. Opin. Ther. Patents (1998) 8(2):143-152. Many of the above cited publications include information on how to make the CRF antagonists described therein. The importance of CRF antagonists is also set out in, e.g., P. Black, Scientific American: “Science & Medicine,” 1995, 2:16-25; T. Lovenberg, et al., Current Pharmaceutical Design, 1995, 1: 305-316; D. T. Chalmers et al., Trends in Pharmacological Sciences, April 1996, pages 166-172; and U.S. Pat. No. 5,063,245. An outline of the activities possessed by CRF antagonists is found in M. J. Owens et al., 1991, Pharm. Rev., 43:425-473.

In particular, CRF antagonists have been described as effective in the treatment of, for example, stress-related illnesses; mood disorders such as depression, including, for example, depression in cancer patients, depression in Parkinson's patients, Postmyocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; chronic fatigue syndrome; dysthymia; pain perception, such as fibromyalgia; gastrointestinal diseases; hemorrhagic stress; ulcers; stress-induced psychotic episodes; fever; diarrhoea; post-operative ileus; colonic hypersensitivity; irritable bowel syndrome; Chron's disease; spastic colon; inflammatory disorders such as rheumatoid arthritis and osteoarthritis; pain; asthma; psoriasis; allergies; osteoporosis; premature birth; hypertension; congestive heart failure; sleep disorders; neurogenerative diseases such as Alzheimer's disease, senile dementia of the Alzheimer's type, multiinfarct dementia, and Huntington's disease; head trauma; ischemic neuronal damage; excitotoxic neuronal damage; epilepsy; stroke; spinal cord trauma; psychosocial dwarfism; euthyroid sick syndrome; syndrome of inappropriate antidiuretic hormone; obesity; infertility; cancer; muscular spams; urinary incontinence; hypoglycemia and immune dysfunctions, including stress-induced immune dysfunctions, immune suppressions, and human immunodeficiency virus infections; stress-induced infections; anxiety disorders, including, for example, generalized anxiety disorder, panic disorder, post-traumatic stress disorder (PTSD), and social anxiety disorder; phobias, including, for example, agoraphobia, social phobia or simple phobias; eating disorders, including, for example, anorexia nervosa or bulimia nervosa; chemical dependencies and addictions, including, for example, addictions to alcohol, cocaine, amphetamine and other psychostimulants, morphine, heroin and other opioid agonists, phenobarbital and other barbiturates, nicotine, and diazepam and other benzodiazepines; drug and alcohol withdrawal symptoms; Parkinson's diseases, including, for example, dementia in Parkinson's disease, neuroleptic-induced parkinsonism or tardive dyskinesias; and headache, including, for example, headache associated with vascular disorders. See, for example, P. Black, Scientific American, 1995, 2:16-25; T. Lovenberg, et al., Current Pharmaceutical Design, 1995, 1:305-316; D. T. Chalmers et al., Trends in Pharmacological Sciences, April 1996, pages 166-172; M. J. Owens et al., Pharm. Rev., 1991,43:425-473; and U.S. Pat. No. 5,063,245.

The present invention is directed to compositions, methods and kits which fulfill the need for simplification of treatment of mood disorders or conditions, psychotic disorders or conditions, or a combination thereof by combining two therapeutic agents. In particular, the compositions contain atypical antipsychotics and corticotropin releasing factor antagonists for the treatment of mood disorders or conditions, psychotic disorders or conditions, or a combination thereof.

### SUMMARY OF THE INVENTION

The present invention is directed to a pharmaceutical compositions for treating, for example, mood disorders or conditions, psychotic disorders or conditions, or,a combination thereof, in a mammal such as a human, the composition comprising (a) an atypical antipsychotic, a prodrug thereof or a pharmaceutically acceptable salt of the atypical antipsychotic or prodrug thereof, (b) a corticotropin releasing factor antagonist, a prodrug thereof, or pharmaceutically acceptable salt of said corticotropin releasing factor antagonist or prodrug thereof, and optionally (c) a pharmaceutically acceptable vehicle, carrier or diluent.

The present invention is also directed to:
a method for treating one or more disorders or conditions described in the previous paragraph, the method comprising administering to a mammal in need of such treatment components (a) and (b) described in the previous paragraph, wherein (a) and (b) are each optionally and independently administered together with a pharmaceutically acceptable vehicle, carrier or diluent;
a composition for treating, for example, a depressive symptom associated with one or more disorders or conditions described in the previous paragraph, the composition comprising components (a), (b), and optionally (c) described in the previous paragraph, wherein the symptom is selected from the group consisting of depressed mood, irritability, sad effect, and circadian rhythm alteration;
a method for treating a depressive symptom associated with one or more disorders or conditions described in the previous paragraph, the method comprising administering to a mammal in need of such treatment components (a) and (b) described in the previous paragraph, wherein (a) and (b) are each optionally and independently administered together with a pharmaceutically acceptable vehicle, carrier or diluent;
a kit comprising an atypical antipsychotic, a prodrug thereof, or pharmaceutically acceptable salt of said atypical antipsychotic or prodrug thereof in a first unit dosage form; a corticotropin releasing factor antagonist, a prodrug thereof, or pharmaceutically acceptable salt of said atypical antipsychotic or prodrug thereof; in a second unit dosage form; and a container;
a kit for achieving, for example, a therapeutic effect for one or more disorders or conditions described in the previous paragraph, the kit comprising a pharmaceutical composition comprising a corticotropin releasing factor antagonist, a prodrug thereof, or pharmaceutically acceptable salt of said corticotropin releasing factor antagonist or prodrug thereof, a package containing the composition, and a package insert that is optionally integral with the package, wherein it is stated on the package insert that the pharmaceutical composition is to be administered to the mammal simultaneously or in a specifically timed manner with a pharmaceutical composition containing an atypical antipsychotic, a prodrug thereof, or pharmaceutically acceptable salt of said atypical antipsychotic or prodrug thereof; and
a kit for achieving, for example, a therapeutic effect for one or more disorders or conditions described in the previous paragraph, the kit comprising a pharmaceutical composition comprising an atypical antipsychotic, a prodrug thereof, or pharmaceutically acceptable salt of said atypical antipsychotic, or prodrug thereof, a package containing the composition, and a package insert that is optionally integral with the package, wherein it is stated on the package insert that the pharmaceutical composition is to be administered to the mammal simultaneously or in a specifically timed manner with a pharmaceutical composition containing corticotropin releasing factor antagonist, a prodrug thereof, or pharmaceutically acceptable salt of said corticotropin releasing factor antagonist or prodrug thereof.

A further feature of the present invention is that the amount of the atypical antipsychotic used to treat mood disorders or conditions, psychotic disorders or conditions, or a combination thereof is a lower amount than the amount of the atypical antipsychotic used to treat such disorders or conditions when the atypical antipsychotic is used in the absence of another therapeutically active agent. The reduced amount of the atypical antipsychotic permits better management of drug-related toxicity and side effects. The amount of the atypical antipsychotic in the composition of the invention that is used to achieve the same or a similar psychotropic effect as when the atypical antipsychotic is used in the absence of another therapeutically active agent is lower by about 25-90%, for example, about 40-80% and typically about 50-70%. The reduction in amount of the atypical antipsychotic required may depend on the amount of the corticotropin releasing factor antagonist.

The term ‘mood disorders’ refers to a group of heterogeneous illnesses including unipolar (depressive) and bipolar (manic-depressive) disorders that are characterized by pervasive mood disturbances, psychomotor dysfunction, and vegetative symptoms. Depression and elation are the core affective components, but anxiety and irritability are equally common, explaining the continued popularity of the broader rubric “affective disorders”, the previous official designation. Types of depression that may be treated by the compositions, methods and kits of this invention include, inter alia: depression in cancer patients, depression in Parkinson's patients, Postmyocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-resistant depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, and major depression with dysthymia. Types of anxiety that may be treated by the compositions, methods and kits of this invention include, inter alia: generalized anxiety disorder, panic disorder, post-traumatic stress disorder (PTSD), social anxiety disorder, treatment-resistant obsessive-compulsive disorder, treatment-resistant anxiety disorder, treatment-resistant generalized anxiety disorder, treatment-resistant post-traumatic stress disorder.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; brief psychotic disorder; shared psychotic disorder; psychotic disorder due to a general medical condition; substance-induced psychotic disorder, for example psychosis induced by alcohol, amphetamine, cannabis, cocaine, hallucinogens, inhalants, opioids, or phencyclidine; personality disorder of the paranoid type; personality disorder of the schizoid type; psychotic disorder not otherwise specified.

Schizophrenia as used herein refers to a disorder that lasts for at least 6 months and includes at least one month of active-phase symptoms (i.e., two [or more] of the following: delusions, hallucinations, disorganized speech, grossly disorganized or catatonic behavior, negative symptoms) (Diagnostic and Statistical Manual of Mental Disorders, DSM-IV-TR, 4^{th} ed, American Psychiatric Assoc., Washington, D.C., 2002).

Schizophreniform disorder is defined as a disorder characterized by a symptomatic presentation that is equivalent to schizophrenia except for its duration (i.e., the disturbance lasts from 1 to 6 months) and the absence of a requirement that there be a decline in functioning (Diagnostic and Statistical Manual of Mental Disorders, DSM-IV-TR, 4^{th} ed, American Psychiatric Assoc., Washington, D.C., 2002).

Schizoaffective disorder is defined as a lifetime pattern of social and interpersonal deficits characterized by an inability to form close interpersonal relationships, eccentric behavior, and mild perceptual distortions.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Examples of symptoms of schizophrenia and schizophreniform and schizoaffecctive disorders also include disorganized speech, affective flattening, alogia, anhedonia, inappropriate affect, dysphoric mood (in the form of, for example, depression, anxiety or anger), and some indications of cognitive dysfunction.

Delusional disorder as referred to herein is characterized by at least 1 month of nonbizarre delusions without other active-phase symptoms of schizophrenia. (Diagnostic and Statistical Manual of Mental Disorders, DSM-IV-TR, 4^{th} ed, American Psychiatric Assoc., Washington, D.C., 2002).

Brief psychotic disorder is a disorder that lasts more than 1 day and remits by 1 month. (Diagnostic and Statistical Manual of Mental Disorders, DSM-IV-TR, 4^{th} ed, American Psychiatric Assoc., Washington, D.C., 2002).

Shared psychotic disorder is characterized by the presence of a delusion in an individual who is influenced by someone else who has a longer-standing delusion with similar content. (Diagnostic and Statistical Manual of Mental Disorders, DSM-IV-TR, 4^{th} ed, American Psychiatric Assoc., Washington, D.C., 2002).

Psychotic disorder due to a general medical condition is characterized by psychotic symptoms judged to be a direct physiological consequence of a general medical condition. (Diagnostic and Statistical Manual of Mental Disorders, DSM-IV-TR, 4^{th} ed, American Psychiatric Assoc., Washington, D.C., 2002).

Psychotic disorder not otherwise specified is a psychotic presentation that does not meet the criteria for any of the specific psychotic disorders defined in the DSM-IV-TR (American Psychiatric Assoc., Washington, D.C., 2002).

The present invention is also useful to treat other disorders that may present psychotic symptoms as associated features such as dementia of the Alzheimer's type; substance-induced delirium; and major depressive disorder with psychotic features.

Other disorders and conditions that may be treated by the compositions, methods and kits of this invention include, inter alia:
phobias, including agoraphobia, social phobia and simple phobias;
sexual dysfunction, including premature ejaculation;
eating disorders, including anorexia nervosa and bulimia nervosa;
chemical dependencies, including addictions to alcohol, cocaine, heroin, phenolbarbitol, nicotine and benzodiazepines;
memory disorders, including dementia, amnestic disorders, and age-related cognitive decline (ARCD);
Parkinson's diseases, including dementia in Parkinson's disease, neuroleptic-induced parkinsonism and tardive dyskinesias;
endocrine disorders, including hyperprolactinaemia;
vasospasm, including a vasospasm in the cerebral vasculature;
gastrointestinal tract disorders, including gastrointestinal tract disorders involving changes in motility and secretion;
cancer, including small cell lung carcinoma; and
headache, including headache associated with vascular disorders. The compositions, methods and kits of the present invention may also used for treating or preventing osteoporosis or frailty associated with aging or obesity, cardiovascular or heart related disease, in particular hypertension, tachycardia, and congestive heart failure, accelerating bone fracture repair, attenuating protein catabolic response after a major operation, reducing cachexia and protein loss due to chronic illness, accelerating wound healing, or accelerating the recovery of burn patients or of patients having undergone major surgery.

The meanings attributed to the different types and subtypes of mood disorders not defined herein are as stated in DSM-IV-TR under depressive disorders (“unipolar depression”) and bipolar disorders, generalized anxiety disorder, and more specific anxiety disorders such as agoraphobia, panic disorder and social phobia, obsessive-compulsive disorder and post traumatic stress disorder (PTSD), the contents of which are incorporated by reference herein. (Diagnostic and Statistical Manual of Mental Disorders”, 4^{th} ed, American Psychiatric Assoc., Washington, D.C., 2002, p. 345-484). Similarly, the meanings attributed to the different types and subtypes of and psychotic disorders are as stated in DSM-IV-TR.

The methods of this invention also encompass treating the diseases or conditions described herein by the co-administration of two separate pharmaceutical compositions. In this latter embodiment, a first composition comprises a CRF antagonist, and a second composition comprises an atypical antipsychotic. These first and second compositions are preferably co-administered either simultaneously, or in a specifically timed manner.

The term “affective disorder” as used herein is interchangeable with the term “mood disorders” and refers to disorders that are characterized by changes in mood as the primary clinical manifestation, for example, depression.

The term “treatment-resistant” is used herein to define a condition wherein a patient having that condition does not respond to treatment with at least one antidepressant over a period of at least six weeks. For example, the term “treatment-resistant” may define a condition wherein a patient having that condition does not respond to treatment with two or more antidepressants over a period of six to eight weeks.

The term “prodrug” refers to compounds that are drug precursors which, following administration, release the drug in vivo via some chemical or physiological process (e.g., a prodrug on being brought to the physiological pH is converted to the desired drug form). A prodrug of any or all of the compounds (i.e., a CRF antagonist, or an atypical antipsychotic) may be used in the methods, kits, and compositions of the instant invention. In general, prodrugs are functional derivatives of these compounds which are readily convertible in vivo. Conventional procedures for the selection and preparation of suitable prodrug derivatives are described, for example, in Design of Prodrugs, ed. H. Bundgaard, Elsevier, 1985 and can be achieved using methods well known to those skilled in the art. All such prodrugs are within the scope of the combinations, pharmaceutical compositions, methods and kits of this invention.

Upon cleavage, exemplary prodrugs release the corresponding free acid (where applicable), and such hydrolyzable ester-forming residues of the prodrugs of this invention include but are not limited to carboxylic acid substituents wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as N,N-dimethylaminoethyl), carbamoyl-(C₁-C₂)alkyl, N,N-di(C₁-C₂)-alkylcarbamoyl-(C₁-C₂)alkyl, piperidino-, pyrrolidino-, or morpholino(C₂-C₃)alkyl, and the like.

Other exemplary prodrugs (where applicable) are derivatives of an alcohol of the compounds used in this invention wherein the free hydrogen of a hydroxyl substituent is replaced by (C1-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxy-carbonylamino-methyl, succinoyl, (C₁-C₆)alkanoyl, α-amino(C₁-C₄)alkanoyl, arylacetyl, α-aminoacyl, α-aminoacyl-.α-aminoacyl wherein said α-aminoacyl moieties are independently any of the naturally occurring L-amino acids found in proteins, —P(O)(OH)₂, —P(O)(O(C₁-C₆)alkyl)₂, glycosyl (the radical resulting from detachment of the hydroxyl of the hemiacetal of a carbohydrate), or the like.

Atypical antipsychotics which may be used in the present invention include olanazapine, clozapine, aripiprazole, quetiapine, amisulpride, risperidone, sertindole; the compounds represented by the structure A 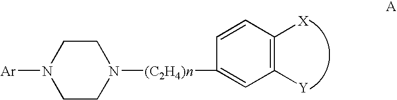
wherein Ar is benzoisothiazolyl or an oxide or dioxide thereof each optionally substituted by one fluoro, chloro, trifluoromethyl, methoxy, cyano, or nitro;
n is 1 or 2;
and X and Y together with the phenyl to which they are attached form benzothiazolyl; 2-aminobenzothiazolyl; benzoisothiazolyl; indazolyl; 2-hydroxyindazolyl; indolyl; oxindolyl optionally substituted by one to three of (C₁-C₃)alkyl, or one of chloro, fluoro or phenyl, said phenyl optionally substituted by one chloro or fluoro; benzoxazolyl; 2-aminobenzoxazolyl; benzoxazolonyl; 2-aminobenzoxazolinyl; benzothiazolonyl; bezoimidazolonyl; or benzotriazolyl;
and the compounds represented by the structure B: 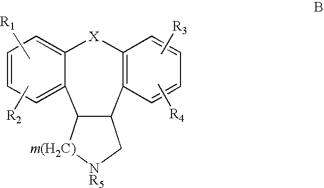
   or pharmaceutically acceptable salts thereof, wherein
R₁, R₂, R₃ and R₄ each represent hydrogen, hydroxy, halogen, a C₁₋C₆alkyl group, an alkoxy or alkylthio group in which the alkyl group contains 1-6 carbon atoms, or a trifluoromethyl group,
R₅ represents hydrogen, a C₁₋C₆ alkyl group carbon atoms or an aralkyl group with 7-10 carbon atoms,
m is 1 or 2,
X represents oxygen, sulphur, the group —N(R₆)— or the group —CH₂—, and
R₆ represents hydrogen or a C₁-C₄ alkyl group.

In an exemplary embodiment, pharmaceutical combinations and methods of treatment include ziprasidone as the atypical antipsychotic of Structure A. Ziprasidone (5-[2-[4-(1,2-benzisothiazol-3-yl )piperazin-1-yl]ethyl]-6-chloroindolin-2-one hydrochloride hydrate) is a benzisothiazolyl piperazine-type atypical antipsychotic with in vitro activity as a 5-HT_{1A} receptor agonist and an inhibitor of serotonin and norepinephrine reuptake (See e.g. U.S. Pat. No. 4,831,031). The postsynaptic 5-HT_{1A} receptor has been implicated in both depressive and anxiety disorders (N M Barnes, T Sharp, 38 Neuropharmacology 1083-152,1999). Oral bioavailability of ziprasidone taken with food is approximately 60%, half-life is approximately 6-7 hours, and protein binding is extensive.

Ziprasidone is efficacious for the treatment of patients with schizophrenia and schizomood disorders, refractory schizophrenia, cognitive impairment in schizophrenia, affective and anxiety symptoms associated with schizoaffective disorder and bipolar disorder. Ziprasidone is considered a safe and efficacious atypical antipsychotic (Charles Caley & Chandra Cooper, 36 Ann. Pharmacother. 839-51, 2002).

The present invention is useful in treating mental disorders and conditions the treatment of which is facilitated by the administration of ziprasidone. Thus, the present invention has application where ziprasidone use is indicated as, e.g., in U.S. Pat. Nos. 6,245,766; 6,245,765; 6,387,904; 5,312,925; 4,831,031; and European EP 0901789 published Mar. 17, 1999, all of which are incorporated herein by reference.

In an exemplary embodiment, pharmaceutical combinations and methods of treatment include trans-5-chloro-2-methyl-2,3,3a,12b-tetrahydro-1H-dibenz[2,3:6,7]oxepino-[4,5-c]pyrrole as the atypical antipsychotic of Structure B. Trans-5-chloro-2-methyl-2,3,3a,12b-tetrahydro-1H-dibenz[2,3:6,7]oxepino-[4,5-c]pyrrole is also referred to herein as asenapine. Asenapine is described, for example, in U.S. Pat. No. 4,145,434. A method of treatment of mental disorders such as psychosis and schizophrenia is described in U.S. Pat. No. 5,763,476. A method of synthesis of asenapine and its maleate salt is shown in Scheme I below.

Other atypical antipsychotics which can be used in the present invention include, but are not limited to, the compounds described in the following paragraphs.

Olanzapine, 2-methyl-4-(4-methyl-1-piperazinyl)-10H-thieno[2,3-b][1,5]benzo-diazepine, is a known compound and is described in U.S. Pat. No. 5,229,382 as being useful for the treatment of schizophrenia, schizophreniform disorder, acute mania, mild anxiety states, and psychosis.

Clozapine, 8-chloro-11-(4-methyl-1-piperazinyl)-5H-dibenzo[b,e][1,4]diazepine, is described in U.S. Pat. No. 3,539,573. Clinical efficacy in the treatment of schizophrenia is described (Hanes, et al., Psychopharmacol. Bull., 24, 62 (1988)).

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, and its use in the treatment of psychotic diseases are described in U.S. Pat. No. 4,804,663

Sertindole, 1-[2-[4-[5-chloro-1-(4-fluorophenyl)-1H-indol-3-yl]-1-piperidinyl]ethyl]imidazolidin-2-one, is described in U.S. Pat. No. 4,710,500. Its use in the treatment of schizophrenia is described in U.S. Pat. Nos. 5,112,838 and 5,238,945.

Quetiapine, 5-[2-(4-dibenzo[b,f][1,4]thiazepin-11-yl -1-piperazinyl)ethoxy]ethanol, and its activity in assays which demonstrate utility in the treatment of schizophrenia are described in U.S. Pat. No. 4,879,288. Quetiapine is typically administered as its (E)-2-butenedioate (2:1) salt.

Aripiprazole, 7-{4-[4-(2,3-dichlorophenyl)-1-piperazinyl]-butoxy}-3,4-dihydro carbostyril or 7-{4-[4-(2,3-dichlorophenyl)-1-piperazinyl]-butoxy}-3,4-dihydro -2(1H)-quinolinone, is an atypical antipsychotic agent used for the treatment of schizophrenia and described in U.S. Pat. No. 4,734,416 and U.S. Pat. No. 5,006,528.

Amisulpride is an atypical antipsychotic agent described in U.S. Pat. No. 4,401,822.

The CRF antagonist may be, for example,

I. a compound of the following formula, described in WO 94/13677: 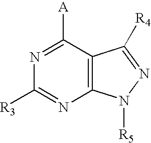
and the pharmaceutically acceptable acid addition salts thereof, wherein
A is NR₁R₂, CR₁R₂R₁₁, or C(═CR₁R₁₂)R₂, NHCR₁R₂R₁₁, OCR₁R₂R₁₁, SCR₁R₂R₁₁, NHNR₁R₂, CR₂R₁₁NHR₁, CR₂R₁₁OR₁, CR₂R₁₁SR₁ or C(O)R₂;
R₁ is hydrogen, or C₁-C₆ alkyl which may be substituted by one or two substituents R₆ independently selected from the group consisting of hydroxy, fluoro, chloro, bromo, iodo, C₁-C₆ alkoxy, O—C(O)—(C₁-C₆ alkyl), O—C(O)—N(C₁-C₄ alkyl)(C₁-C₂ alkyl); amino, NH(C₁-C₄ alkyl), S(C₁-C₆ alkyl), OC(O)NH(C₁-C₄ alkyl), N(C₁-C₂ alkyl)C(O)(C₁-C₄ alkyl), NHC(O)(C₁-C₄ alkyl, COOH, CO(C₁-C₄ alkyl), C(O)NH(C₁-C₄ alkyl), C(O)N(C₁-C₄ alkyl)(C₁-C₂ alkyl), SH, CN, NO₂, SO(C₁-C₄ alkyl); SO₂(C₁-C₄ alkyl), SO₂NH(C₁-C₄ alkyl), SO₂N(C₁-C₄ alkyl)(C₁-C₂ alkyl), and said C₁-C₆ alkyl may have one or two double or triple bonds;
R² is C₁-C₁₂ alkyl, aryl or (C₁-C₁₀ alkylene)aryl wherein said aryl is phenyl, naphthyl, thienyl, benzothienyl, pyridyl, quinolyl, pyrazinolyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, azaindolyl, oxazolyl, or benzoxazolyl; 3- to 8-membered cycloalkyl or (C₁-C₆ alkylene) cycloalkyl, wherein said cycloalkyl may have one or two of O, S or N-Z, wherein Z is hydrogen, substituted, independently, for one or two carbons of said cycloalkyl, C₁-C₄ alkyl, benzyl or C₁-C₄ alkanoyl, wherein R² may be substituted independently by from one to three of chloro, fluoro, or C₁-C₄ alkyl, or one of hydroxy, bromo, iodo, C₁-C₆ alkoxy, OC(O)(C₁-C₆ alkyl), O—C—N(C₁-C₄ alkyl)(C₁-C₂ alkyl), S(C₁-C₆ alkyl), NH₂, NH(C₁-C₂ alkyl), N(C₁-C₄ alkyl) C(O)(C₁-C₄ alkyl), NHC(O)(C₁-C₄ alkyl), COOH, C(O)O(C₁-C₄ alkyl), C(O)NH(C₁-C₄ alkyl), C(O)N(C₁-C₄ alkyl)(C₁-C₂ alkyl), SH, CN, NO₂, SO(C₁-C₄ alkyl), SO₂(C₁-C₄ alkyl), SO₂NH(C₁-C₄ alkyl), SO₂N(C₁-C₄ alkyl)(C₁-C₂ alkyl), and wherein said C₁-C₁₂ alkyl or C₁-C₁₀ alkylene may have one to three double or triple bonds; or
NR₁R₂ or CR₁R₂R₁₁ may form a 4- to 8-membered ring optionally having one or two double bonds or one or two of O, S or N-Z wherein Z is hydrogen, C₁-C₄ alkyl, benzyl, or C₁-C₄ alkanoyl;
R₃ is hydrogen, C₁-C₆ alkyl, fluoro, chloro, bromo, iodo, hydroxy, amino, O(C₁-C₆ alkyl), NH(C₁-C₆ alkyl), N(C₁-C₄ alkyl)(C₁-C₂ alkyl), SH, S(C₁-C₄ alkyl), SO(C₁-C₄ alkyl), or SO₂(C₁-C₄ alkyl), wherein said C₁-C₄ alkyl and C₁-C₆ alkyl may have one or two double or triple bonds and may be substituted by from 1 to 3 R₇ substituents independently selected from the group consisting of hydroxy, amino, C₁-C₃ alkoxy, dimethylamino, diethylamino, methylamino, ethylamino, NHC(O)CH₃, fluoro, chloro or C₁-C₃ thioalkyl;
R₄ is hydrogen, C₁-C₆ alkyl, fluoro, chloro, bromo, iodo, C₁-C₆ alkoxy, amino, NH(C₁-C₆ alkyl), N(C₁-C₆ alkyl) (C₁-C₂ alkyl), SO,(C₁-C₆ alkyl), wherein n is 0, 1 or 2, cyano, hydroxy, carboxy, or amido, wherein said C₁-C₆ alkyls may be substituted by one to three of hydroxy, amino, carboxy, amido, NHC(O)(C₁-C₄ alkyl), NH(C₁-C₄ alkyl), N(C₁-C₄ alkyl)(C₁-C₂ alkyl), C(O)O(C₁-C₄ alkyl), C₁-C₃ alkoxy, C₁-C₃ thioalkyl, fluoro, bromo, chloro, iodo, cyano or nitro;
R₅ is phenyl, naphthyl, thienyl, benzothienyl, pyridyl, quinolyl, pyrazinolyl, pyrimidyl, imidazolyl, furanyl, benzofuranyl, benzothiazolyl, isothiazolyl, benzoisothiazolyl, thiazolyl, isoxazolyl, benzisoxazolyl, benzimidazolyl, triazolyl, pyrazolyl, pyrrolyl, indolyl, pyrrolopyridyl benzoxazolyl, oxazolyl, pyrrolidinyl, thiazolidinyl, piperazinyl, piperidinyl, or tetrazolyl, wherein each one of the above groups may be substituted independently by from one to three of fluoro, chloro, bromo, formyl, C₁-C₆ alkyl, C₁-C₆ alkoxy or trifluoromethyl, or one of hydroxy, iodo, cyano, nitro, amino, cyclopropyl, NH(C₁-C₄ alkyl), N(C₁-C₄ alkyl)(C₁-C₂ alkyl), COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), SO₂(C₁-C₆ alkyl), wherein said C₁-C₄ alkyl and C₁-C₆ alkyl may have one double or triple bond and may be substituted by one or two of fluoro, chloro, hydroxy, amino, methylamino, dimethylamino or acetyl; with the proviso that R₅ is not unsubstituted phenyl;
R₁₁ is hydrogen, hydroxy, fluoro, chloro, COO(C₁-C₂ alkyl), cyano, or CO(C₁-C₂ alkyl); and
R₁₂ is hydrogen or C₁-C₄ alkyl;
   (a) A is not straight chain C₁-C₁₂ alkyl;
   (b) when R₃ is hydrogen, A is benzyl or phenethyl, and R₄ is fluoro, chloro, bromo or iodo, then R₅ is not 5′-deoxy-ribofuranosyl or 5′-amino-5′-deoxy-ribofuranosyl; and
   (c) when R⁵ is phenyl, said phenyl is substituted by two or three substituents.

II. The invention also relates to use of a CRF antagonist of the following formula, described in WO 94/13676: 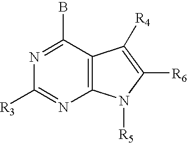
and the acid addition salts thereof, wherein
B is XA wherein X is (CH₂)ₙ in which n is 0, 1 or 2, NH, O, S, N(C₁-C₄ alkyl);
A is NR₁R₂, CR₁R₂R₁₁, or C(═CR₂R₁₂)R₁;
R₁ is hydrogen, or C₁-C₆ alkyl which may be substituted by one or two substituents R₇ independently selected from the group consisting of hydroxy, fluoro, chloro, bromo, iodo, C₁-C₈ alkoxy, O—C(═O)—(C₁-C₆ alkyl), O—C(═O)NH(C₁-C₄ alkyl), O—C(═O)—N(C₁-C₄ alkyl)(C₁-C₂ alkyl), amino, NH(C₁-C₄ alkyl), N(C₁-C₂ alkyl)(C₁-C₄ alkyl), S(C₁-C₆ alkyl), N(C₁-C₄alkyl)C(═O)(C₁-C₄ alkyl), NH(C₁-C₄ alkyl), COOH, C(═O)O(C₁-C₄ alkyl), C(═O)NH(C₁-C₄ alkyl), C(═O)N(C₁-C₄ alkyl)(C₁-C₂ alkyl), SH, CN, NO₂, SO(C₁-C₄ alkyl), SO₂(C₁-C₄ alkyl), SO₂NH(C₁-C₄ alkyl), SO₂N(C₁-C₄ alkyl)(C₁-C₂ alkyl), and said C₁-C₆ alkyl may contain one or two double or triple bonds;
R₂ is C₁-C₁₂ alkyl, aryl or (C₁-C₁₀ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, pyrrolopyridyl, oxazolyl, or benzoxazolyl; 3- to 8-membered cycloalkyl or (C₁-C₆ alkylene) cycloalkyl, wherein said cycloalkyl may contain one or two of O, S or N-Z wherein Z is hydrogen, C₁-C₄ alkyl, benzyl or C₁-C₄ alkanoyl, wherein R₂ may be substituted independently by from one to three of chloro, fluoro, or C₁-C₄ alkyl, or one of hydroxy, bromo, iodo, C₁-C₆ alkoxy, O—C(═O)—(C₁-C₆ alkyl), O—C—N(C₁-C₄ alkyl)(C₁-C₂ alkyl), S(C₁-C₆ alkyl), NH₂, NH(C₁-C₂ alkyl), N(C₁-C₂ alkyl) (C₁-C₄ alkyl), N(C₁-C₄)—C(±O)(C₁-C₄ alkyl), NHC(═O)(C₁-C₄), COOH, C(═O)O(C₁-C₄ alkyl), C(═O)NH(C₁-C₄ alkyl), C(═O)N(C₁-C₄ alkyl)(C₁-C₂ alkyl), SH, CN, NO₂, SO(C₁-C₄ alkyl), SO₂(C₁-C₄ alkyl), SO₂NH(C₁-C₄ alkyl), SO₂N(C₁-C₄ alkyl)(C₁-C₂ alkyl), and wherein said C₁-C₁₂ alkyl or C₁-C₁₀ alkyl may contain one to three double or triple bonds; or
when A is NR₁R₂ or CR₁R₂R₁₁, then R₁ and R₂ taken together with the atom to which they are attached may form a saturated 4- to 8-membered optionally containing one or two double bonds or one or two of O, S or N-Z wherein Z is hydrogen, C₁-C₄ alkyl, or C₁-C₄ alkanoyl;
R₃ is hydrogen, C₁-C₆ alkyl, fluoro, chloro, bromo, iodo, hydroxy, amino, O(C₁-C₆ alkyl), NH(C₁-C₆ alkyl), N(C₁-C₄ alkyl)(C₁-C₂ alkyl), SH, S(C₁-C₄ alkyl), SO(C₁-C₄ alkyl), or SO₂(C₁-C₄ alkyl), wherein said C₁-C₄ alkyl and C₁-C₆ alkyl may contain from one or two double or triple bonds and may be substituted by from 1 to 3 substituents R₈ independently selected from the group consisting of hydroxy, amino, C₁-C₃ alkoxy, dimethylamino, diethylamino, methylamino, ethylamino, NHCH₃, fluoro, chloro or C₁-C₃ thioalkyl;

R1₄ and R₆ are each independently hydrogen, C₁-C₆alkyl, fluoro, chloro, bromo, iodo, C₁-C₆ alkoxy, amino, NH(C₁-C₆ alkyl), N(C₁-C₆ alkyl)(C₁-C₂ alkyl), SOₙ(C₁-C₆ alkyl), wherein n is 0, 1 or 2, cyano, hydroxy, carboxy, or amido, wherein said C₁-C₆ alkyls may be substituted by one to three of hydroxy, amino, carboxy, amido, NHC(═O)(C₁-C₄ alkyl), NH(C₁-C₄ alkyl), N(C₁-C₄ alkyl)(C₁-C₂ alkyl), C(═O)O(C₁-C₄ alkyl), C₁-C₃ alkoxy, C₁-C₃ thioalkyl, fluoro, bromo, chloro, iodo, cyano or nitro;
R₅ is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, azaindolyl, benzoxazolyl, oxazolyl, pyrrolidinyl, thiazolidinyl, morpholinyl, piperidinyl, piperazinyl, tetrazolyl, or 3- to 8-membered cycloalkyl or 9- to 1 2-membered bicycloalkyl, optionally containing one to three of O, S or N-Z wherein Z is hydrogen, C₁-C₄ alkyl, C₁-C₄ alkanoyl, phenyl or phenylmethyl, wherein each one of the above groups may be substituted independently by from one to four of fluoro, chloro, C₁-C₆ alkyl, C₁-C₆ alkoxy or trifluoromethyl, or one of bromo, iodo, cyano, nitro, amino, NH(C₁-C₄ alkyl), N(C₁-C₄)(C₁-C₂ alkyl), COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), SO₂C₁-C₆ alkyl), wherein said C₁-C₄ alkyl and C₁-C₆ alkyl may be substituted by one or two of fluoro, chloro, hydroxy, amino, methylamino, dimethylamino or acetyl; with the proviso that R₅ is not unsubstituted phenyl;
R₁₁ is hydrogen, hydroxy, fluoro, chloro, COO(C₁-C₂ alkyl), cyano, or CO(C₁-C₂ alkyl); and
R₁₂ is hydrogen or C₁-C₄ alkyl; with the proviso that (1) when R₅ is 4-bromophenyl, R₃ is hydrogen, and R₄ and R₆ are methyl, then B is not methylamino or ethyl, and (2) when R₅ is 4-bromophenyl, and R₃, R₄ and R₆ are methyl, then B is not 2-hydroxyethylamino.

III. It is also possible to employ a CRF antagonist that has a structure selected from the group shown below, and pharmaceutically acceptable salts and esters thereof, as described in WO 95/33750: 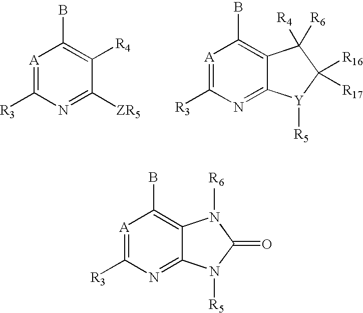
wherein
A is CR₇ or N;
B is NR₁R₂, CR₁R₂R₁₁, C(═CR₂R₁₂)R₁, NHCHR₁R₂, OCHR₁R₂, SCHR₁R₂, CHR₂OR₁₂, CHR₂SR₁₂, C(S)R₂ or C(O)R₂;
Y is CH or N;
Z is NH, O, S, N (C₁-C₂ alkyl), or CR₁₃R₁₄, wherein R₁₃ and R₁₄ are each independently hydrogen, trifluoromethyl, or C₁-C₄ alkyl, or one of R₁₃ and R₁₄ may be cyano, chloro, bromo, iodo, fluoro, hydroxy, O(C₁-C₂ alkyl), amino, NH(C₁-C₂ alkyl), or CR₁₃R₁₄ may be C═O or cyclopropyl;
R₁ is C₁-C₆ alkyl which may be substituted by one or two substituents R₈ independently selected from the group consisting of hydroxy, fluoro, chloro, bromo, iodo, C₁-C₄ alkoxy, O—CO—(C₁-C₄ alkyl), O—CO—NH(C₁-C₄ alkyl), O—CO—N(C₁-C₄ alkyl)(C₁-C₂ alkyl), NH(C₁-C₄ alkyl), N(C₁-C₂ alkyl)(C₁-C₄ alkyl), S(C₁-C₄ alkyl), N(C₁-C₄alkyl)CO(C₁-C₄ alkyl), NHCO(C₁-C₄alkyl), COO(C₁-C₄ alkyl), CONH(C₁-C₄ alkyl), CON(C₁-C₄ alkyl)(C₁-C₂ alkyl), S(C₁-C₄ alkyl), CN, NO₂, SO(C₁-C₄ alkyl), SO₂(C₁-C₄ alkyl), and said C₁-C₆ alkyl or C₁-C₄ alkyl may contain one double or triple bond;
R₂ is C₁-C₁₂ alkyl, aryl or (C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, or benzoxazolyl; 3- to 8-membered cycloalkyl or (C₁-C₆ alkylene)cycloalkyl, wherein said cycloalkyl may contain one or two of O, S or N—R₉ wherein R₉ is hydrogen, or C,-C₄ alkyl, wherein the above defined R₂ may be substituted independently by from one to three of chloro, fluoro, or C₁-C₄ alkyl, or one of bromo, iodo, C₁-C₆ alkoxy, O—CO—(C₁-C₆ alkyl), O—CO—N(C₁-C₄ alkyl)(C₁-C₂ alkyl), S(C₁-C₆ alkyl), CN, NO₂, SO(C₁-C₄ alkyl), or SO₂(C₁-C₄ alkyl), and wherein said C₁-C₁₂ alkyl or C₁-C₄ alkylene may contain one double or triple bond; or
NR₁R₂ or CR₁R₂R₁₁ may form a saturated 5- to 8-membered carbocyclic ring which may contain one or two double bonds or one or two of O or S;
R₃ is methyl, ethyl, fluoro, chloro, bromo, iodo, cyano, methoxy, OCF₃, methylthio, methylsulfonyl, CH₂OH or CH₂OCH₃;
R₄ is hydrogen, C₁-C₄ alkyl, fluoro, chloro, bromo, iodo, C₁-C₄ alkoxy, amino, nitro, NH(C₁-C₄ alkyl), N(C₁-C₄ alkyl)(C₁-C₂ alkyl), SOₙ(C₁-C₄ alkyl), wherein n is 0, 1 or 2 cyano, hydroxy, CO(C₁-C₄ alkyl), CHO, or COO(C₁-C₄ alkyl), wherein said C₁-C₄ alkyl may contain one or two double or triple bonds and may be substituted by one or two of hydroxy, amino, carboxy, NHCOCH₃, NH(C₁-C₂ alkyl), N(C₁-C₂ alkyl)₂, COO(C₁-C₄ alkyl), CO(C₁-C₄ alkoxy, C₁-C₃ thioalkyl, fluoro, chloro, cyano or nitro;
R₅ is phenyl, naphthyl, thienyl, benzothienyl, pyridyl, quinolyl, pyrazinyl, pyrimidyl, furanyl, benzofuranyl, benzothiazolyl, or indolyl, wherein each one of the above groups R₅ is substituted independently by from one to three of fluoro, chloro, C₁-C₆ alkyl, or C₁-C₆ alkoxy, or one of hydroxy, iodo, bromo, formyl, cyano, nitro, trifluoromethyl, amino, NH(C₁-C₄ alkyl), N(C₁-C₆)(C₁-C₂ alkyl), COOH, COO(C₁-C₄ alkyl), CO(C₁-C₄ alkyl), SO₂NH(C₁-C₄ alkyl), SO₂(C₁-C₄ alkyl)(C₁-C₂ alkyl), SO₂NH₂, NHSO₂(C₁-C₄ alkyl), S(C₁-C₆ alkyl), or SO₂(C₁-C₄ alkyl, wherein said C₁-C₄ alkyl and C₁-C₆ alkyl may be substituted by one or two of fluoro, hydroxy, amino, methylamino, dimethylamino or acetyl;
R₆ is hydrogen, or C₁-C₆ alkyl, wherein said C₁-C₆ alkyl may be substituted by one hydroxy, methoxy, ethoxy or fluoro;
R₇ is hydrogen, C₁-C₄ alkyl, fluoro, chloro, bromo, iodo, cyano, hydroxy, O(C₁-C₄ alkyl), C(O)(C₁-C₄ alkyl), or C(O)O(C₁-C₄ alkyl), wherein the C₁-C₄ alkyl groups may be substituted with one hydroxy, chloro or bromo, or one to three fluoro;
R₁₁ is hydrogen, hydroxy, fluoro, or methoxy;
R₁₂ is hydrogen or C₁-C₄ alkyl; and
R₁₆ and R₁₇ are each independently hydrogen, hydroxy, methyl, ethyl, methoxy, or ethoxy, except that they are not both methoxy or ethoxy, and CR₄R₆ and CR₁₆R₁₇ each independently may be C═O.

IV. It also possible to employ a CRF antagonist of the following formula, disclosed in WO 95/34563: 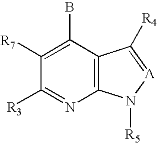
and the pharmaceutically acceptable acid addition salts thereof, wherein
A is N or —CR₆;
B is —NR₁R₂, —CR₁R₂R₁₁, —C(═CR₂R₁₂)R₁, —NHCHR₁R₂, —OCHR₁R₂, —SCHR₁R₂, —CHR₂OR₁₂, —CHR₂SR₁₂, —C(S)R₁ or —C(O)R₁;

R₁ is C₁-C₆ alkyl which may optionally be substituted with one or two substituents independently selected from the group consisting of hydroxy, fluoro, chloro, bromo, iodo, C₁-C₄ alkoxy, —O—CO—(C₁-C₄ alkyl), —O—CO—NH(C₁-C₄ alkyl), —O—CO—N(C₁-C₄ alkyl)(C₁-C₂ alkyl), —NH(C₁-C₄ alkyl), —N(C₁-C₂ alkyl)(C₁-C₄ alkyl), —S(C₁-C₄ alkyl), —N(C₁-C₄alkyl)CO(C₁-C₄ alkyl), —NHCO(C₁-C₄ alkyl), —COO(C₁-C₄ alkyl), —CONH(C₁-C₄ alkyl), —CON(C₁-C₄ alkyl)(C₁-C₂ alkyl), CN, NO₂, ₁—SO(C₁-C₄ alkyl), —SO₂(C₁-C₄ alkyl), and wherein any of the foregoing C₁-C₄ alkyl and C₁-C₆ alkyl groups may optionally contain one carbon-carbon double or triple bond;
R₂ is C₁-C₁₂ alkyl, aryl, —(C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, thiazolyl, isoxazolyl, benzisoxazolyl, benzimidazolyl, triazolyl, pyrazolyl, pyrrolyl, indolyl, oxazolyl, or benzoxazolyl; or 3- to 8-membered cycloalkyl or —(C₁-C₆ alkylene)cycloalkyl, wherein one or two of the ring carbons of said cycloalkyl having at least 4 ring members and the cycloalkyl moiety of said —(C₁-C₆ alkylene)cycloalkyl having at least 4 ring members may optionally be replaced by an oxygen or sulfur atom or by N-Z wherein Z is hydrogen; or C₁-C₄ alkyl, and wherein each of said groups R₂ may optionally be substituted with from one to three substituents independently selected from chloro, fluoro, and C₁-C₄ alkyl, or by one substituent selected from bromo, iodo, C₁-C₆ alkoxy, —O—CO—(C₁-C₆ alkyl), —S(C₁-C₆ alkyl), —COO(C₁-C₄ alkyl), CN, NO₂, —SO(C₁-C₄ alkyl), and —SO₂(C₁-C₄ alkyl), and wherein said C₁-C₁₂ alkyl and the C₁-C₄ alkylene moiety of said —(C₁-C₄ alkylene)aryl may optionally contain one carbon-carbon double or triple bond;
or —NR₁R₂ may form a saturated 5- to 8-membered heterocyclic ring, or —CHR₁R₂ may form a saturated 5- to 8-membered carbocyclic ring, wherein each of these rings may optionally contain one or two carbon-carbon double bonds and wherein one or two of the carbon atoms of each of these rings may optionally be replaced with a sulfur or oxygen atom;
R₃ is C₁-C₄ alkyl, fluoro, chloro, bromo, iodo, —CH₂OH, —CH₂OCH₃, —O(C₁-C₃ alkyl), ₁—S(C₁-C₃ alkyl), or —SO₂(C₁-C₃ alkyl), wherein said C₁-C₃ alkyl may optionally contain one carbon-carbon double or triple bond;
R₄ is hydrogen, C₁-C₆ alkyl, fluoro, chloro, bromo, iodo, C₁-C₄ alkoxy, amino, —NHCH₃, —N(CH₃)₂, —CH₂OH, —CH₂OCH₃, or —SOₙ(C₁-C₄ alkyl), wherein n is 0, 1 or 2, cyano, hydroxy, —CO(C₁-C₄ alkyl),—CHO, or —COO(C₁-C₄ alkyl) wherein the C₁-C₄ alkyl moieties in the foregoing R₄ groups may optionally contain one carbon-carbon double or triple bond;
R₅ is phenyl, naphthyl, thienyl, benzothienyl, pyridyl, pyrimidyl, benzofuranyl, pyrazinyl or benzothiazolyl, wherein each one of said groups R₅ may optionally be substituted with from one to three substituents independently selected from fluoro, chloro, C₁-C₆ alkyl and C₁-C₆ alkoxy, or by one substituent selected from iodo, hydroxy, bromo, formyl, cyano, nitro, amino, trifluoromethyl, —NH(C₁-C₄ alkyl), —N(C₁-C₆)(C₁-C₂ alkyl), —COO(C₁-C₄ alkyl), —CO(C₁-C₄ alkyl), —COOH, —SO₂NH(C₁-C₄ alkyl), —SO₂N(C₁-C₄ alkyl)(C₁-C₂ alkyl), —SO₂NH₂, —NHSO₂(C₁-C₄ alkyl), —S(C₁-C₆ alkyl) and —SO₂(C₁-C₆ alkyl), wherein each of said C₁-C₄ alkyl and C₁-C₆ alkyl moieties in the foregoing R⁵ groups may optionally be substituted with one to three fluorine atoms;
R₆ is hydrogen, C₁-C₄ alkyl, fluoro, chloro, bromo, iodo, —CH₂OH, —CH₂OCH₃, or C₁-C₄ alkoxy;
R₇ is hydrogen, C₁-C₄ alkyl, fluoro, chloro, bromo, iodo, —O(C₁-C₄ alkyl), cyano, —CH₂OH, —CH₂O(C₁-C₂ alkyl), —CO(C₁-C₂ alkyl), or —COO(C₁-C₂ alkyl);
R₁₁ is hydrogen, hydroxy, fluoro, or methoxy; and
R₁₂ is hydrogen or C₁-C₄ alkyl;
with the proviso that when A is N, then: (a) B is not unsubstituted alkyl; (b) R₅ is not unsubstituted phenyl or monosubstituted phenyl; and (c) R₃ is not unsubstituted alkyl;
or a pharmaceutically acceptable salt of such compound.

V. In another embodiment, the CRF antagonist is of the following formula, disclosed in EP 778277: 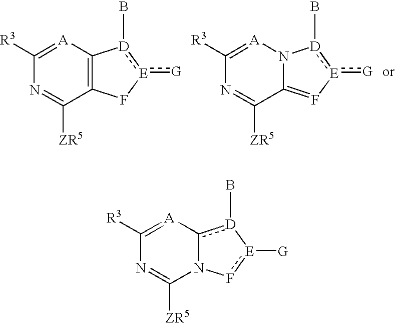
or a pharmaceutically acceptable salt thereof, wherein
the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or —COR²;
D is nitrogen and is single bonded to all atoms to which it is attached, or D is carbon and is either double bonded to E in formulas I and II or double bonded to the adjacent carbon atom common to both fused rings in formula III, or D is CH and is single bonded to E in formulas I and II;
E is nitrogen, CH or carbon;
F is oxygen, sulfur, CHR⁴ or NR⁴ when it is single bonded to E and F is nitrogen or CR⁴ when it is double bonded to E;
G, when single bonded to E, is hydrogen, C₁-C₄ alkyl, —S(C₁-C₄ alkyl), —O(C₁-C₄ alkyl), NH₂, —NH(C₁-C₄ alkyl) or —N(C₁-C₂ alkyl)(C₁-C₄ alkyl), wherein each of the C₁-C₄ alkyl groups of G may optionally be substituted with one hydroxy, —O(C₁-C₂ alkyl) or fluoro group; G, when double bonded to E, is oxygen, sulfur or NH; and G, when E is nitrogen and double bonded to D or F, is absent;
R¹ is hydrogen, C₁-C₆ alkyl optionally substituted with one or two substituents R⁸ independently selected from hydroxy, fluoro, chloro, bromo, iodo, C₁-C₄ alkoxy, CF₃, —C(═O)O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl) may optionally and independently be replaced by an oxygen or sulfur atom or by NZ² wherein Z² is selected from hydrogen, C₁-C₄ alkyl, benzyl and C₁-C₄ alkanoyl, and wherein each of the foregoing R² groups may optionally be substituted with from one to three substituents independently selected from chloro, fluoro, hydroxy and C₁-C₄ alkyl, or with one substituent selected from bromo, iodo, C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a saturated 3 to 8 membered carbocyclic ring which may optionally contain from one to three double bonds and wherein one or two of the ring carbon atoms of such 5 to 8 membered rings may optionally and independently be replaced by an oxygen or sulfur atom or by NZ³ wherein Z³ is hydrogen, C₁-C₄ alkyl, benzyl or C₁-C₄ alkanoyl;
R³ is hydrogen, C₁-C₄ alkyl, —O(C₁-C₄ alkyl), chloro, fluoro, bromo, iodo, —CN, —S(C₁-C₄ alkyl) or —SO₂(C₁-C₄ alkyl) wherein each of the (C₁-C₄ alkyl) moieties in the foregoing R³ groups may optionally be substituted with one substituent R⁹ selected from hydroxy, fluoro and (C₁-C₂ alkoxy);
each R⁴ is, independently, hydrogen, (C₁-C₆ alkyl), fluoro, chloro, bromo, iodo, hydroxy, cyano, amino, nitro, —O(C₁-C₄ alkyl), —N(C₁-C₄ alkyl)(C₁-C₂ alkyl), —S(C₁-C₄ alkyl), —SO(C₁-C₄ alkyl), —SO₂(C₁-C₄)alkyl, —CO(C₁-C₄ alkyl), —C(═O)H or —C(═O)O(C₁-C₄alkyl), wherein each of the (C₁-C₆ alkyl) and (C₁-C₄ alkyl) moieties in the foregoing R⁴ groups may optionally contain one or two double or triple bonds and may optionally be substituted with one or two substituents independently selected from hydroxy, amino, C₁-C₃ alkoxy, dimethylamino, methylamino, ethylamino, —NHC(═O)CH₃, fluoro, chloro, C₁-C₃ thioalkyl, —CN, —COOH, —C(═O)O(C₁-C₄ alkyl), —C(═O)(C₁-C₄ alkyl) and —NO₂;
R⁵ is phenyl, naphthyl, thienyl, benzothienyl, pyridyl, quinolyl, pyrazinyl, furanyl, benzofuranyl, benzothiazolyl, benzisothiazolyl, benzisoxazolyl, benzimidazolyl, indolyl, benzoxazolyl or C₃-C₈ cycloalkyl wherein one or two of the carbon atoms of said cycloalkyl rings that contain at least 5 ring members may optionally and independently be replaced by an oxygen or sulfur atom or by NZ⁴ wherein Z⁴ is hydrogen, C₁-C₄ alkyl or benzyl; and wherein each of the foregoing R⁵ groups is substituted with from one to four substituents R¹² wherein one to three of said substituents may be selected, independently, from chloro, C₁-C₆ alkyl and —O(C₁-C₆ alkyl) and one of said substituents may be selected from bromo, iodo, formyl, —CN, —CF₃, —NO₂, —NH₂, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, C₁-C₄ alkyl, halo, cyano, hydroxy, —O(C₁-C₄ alkyl) —C(═O)(C₁-C₄ alkyl), —C(═O)O(C₁-C₄alkyl), —OCF₃, —CF₃, —CH₂OH, —CH₂O(C₁-C₄ alkyl);
R¹⁰ is hydrogen, hydroxy, methoxy or fluoro;
R¹¹ is hydrogen or C₁-C₄ alkyl; and
Z is NH, oxygen, sulfur, —N(C₁-C₄ alkyl), —NC(═O)(C₁-C₂ alkyl), NC(═O)O(C₁-C₂alkyl) or CR¹³R¹⁴ wherein R¹³ and R¹⁴ are independently selected from hydrogen, trifluoromethyl and methyl with the exception that one of R¹³ and R¹⁴can be cyano;
with the proviso that: (a) in the five membered rings of structures I, II and III, there can not be two double bonds adjacent to each other; and (b) when R⁴ is attached to nitrogen, it is not halo, cyano or nitro;
or a pharmaceutically acceptable salt of such compound.

VI. The CRF antagonist can also be of the following formula, disclosed in WO 98/05661: 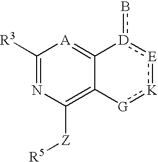
wherein the dashed lines represent optional double bonds;
A is nitrogen or CR⁷;
B is —NR¹R², —CR¹R²R¹⁰, —C(═CR²R¹¹)R¹, —NHCR₁R²R¹⁰, —OCR¹R²R¹⁰, —SCR¹R²R¹⁰, —CR²R¹⁰NHR¹, —CR²R¹⁰OR¹, —CR²R¹⁰SR¹ or —COR², and is single bonded to D; or B is —CR¹R², and is double bonded to D and D is carbon;
D is nitrogen or CR⁴ and is single bonded to all atoms to which it is attached, or D is carbon and is double bonded to E or double bonded to B;
E is oxygen, nitrogen, sulfur, C═O, C═S, CR⁶R², NR⁶ or CR^{6;} or E is a two atom spacer, wherein one of the atoms is oxygen, nitrogen, sulfur, C═O, C═S, CR⁶R¹², NR⁶ or CR⁶, and the other is CR⁶R¹² or CR⁹;
K and G are each, independently, C═O, C═S, sulfur, oxygen, CHR⁸ or NR⁸ when single bonded to both adjacent ring atoms, or nitrogen or CR⁸ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;

R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ³ wherein Z³ is hydrogen or C₁-C₄ alkyl;
R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);
R⁴ is hydrogen, C₁-C₂ alkyl, hydroxy or fluoro;
each R⁶, R⁸ and R⁹ that is attached to a carbon atom is selected, independently, from hydrogen, C₁-C₂ alkyl, fluoro, chloro, bromo, iodo, hydroxy, hydroxymethyl, formyl, trifluoromethyl, cyano, amino, nitro, —O(C₁-C₂ alkyl), —N(C₁-C₂ alkyl)(C₁-C₂ alkyl), —S(C₁-C₂ alkyl), —CO(C₁-C₂ alkyl), —C(═O)H or —C(═O)O(C₁-C₂ alkyl), wherein each of the C₁-C₂ alkyl moieties in the foregoing R⁶, R⁸, and R⁹ groups may optionally contain one double or triple bond; and each R⁶, R⁸, and R⁹ that is attached to a nitrogen atom is selected, independently, from hydrogen and C₁-C₄ alkyl;
R⁵is substituted phenyl, naphthyl, pyridyl or pyrimidyl, wherein each of the foregoing R⁵ groups is substituted with from two to four substituents R¹⁵, wherein from one to three of said substituents may be selected, independently, from chloro, C₁-C₆ alkyl, —O(C₁-C₆ alkyl) and —(C₁-C₆alkylene)O(C₁-C₆alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo (e.g., chloro, fluoro, iodo or bromo), hydroxy, methoxy, —C(═O)(C₁-C₂ alkyl), —C(═O)O(C₁-C₂ alkyl), trifluoromethoxy, hydroxymethyl, trifluoromethyl or formyl;
R¹⁰ is hydrogen, hydroxy, methoxy or fluoro;
R¹¹ is hydrogen or C₁-C₄ alkyl;
R¹² is, hydrogen or methyl; and
Z is NH, oxygen, sulfur, —N(C₁-C₄ alkyl), or CR¹³R¹⁴ wherein R¹³ and R¹⁴ are independently selected from hydrogen, and methyl with the exception that one of R¹³ and R¹⁴ may optionally be cyano;
with the proviso that: (a) in the six or seven membered rings of structures in formula I, there can not be two double bonds adjacent to each other; and (b) when D is carbon and is double bonded to B, then B is CR¹R²;
or a pharmaceutically acceptable salt of such compound.

VII. The CRF antagonist can also be of the following formula, disclosed in WO 98/08847: 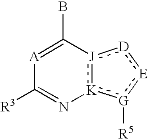
or a pharmaceutically acceptable salt thereof, wherein
the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR²R²OR¹⁰, —CR²R¹⁰SR¹ or —COR²;
J and K are each independently nitrogen or carbon and both J and K are not nitrogens;
D and E are each selected, independently, from nitrogen, CR⁴, C═O, C═S, sulfur, oxygen, CR⁴R⁶ and NR⁸;
G is nitrogen or carbon;
the ring containing D, E, G, K, and J in formula I may be a saturated or unsaturated 5-membered ring and may optionally contain one or two double bonds and may optionally contain from one to three heteroatoms in the ring and may optionally have one or two C═O or C═S groups;
R¹ is C₁-C₆ alkyl optionally substituted with one or two substituents independently selected from hydroxy, fluoro, chloro, bromo, iodo, —O—(C₁-C₄ alkyl), CF₃, —C(═O)O—(C₁-C₄alkyl), —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl),(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₈ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl) may optionally and independently be replaced by an oxygen or sulfur atom or by NZ² wherein Z² is selected from hydrogen, C₁-C₄ alkyl, benzyl and C₁-C₄ alkanoyl, and wherein each of the foregoing R² groups may optionally be substituted with from one to three substituents independently selected from chloro, fluoro, hydroxy and C₁-C₄ alkyl, or with one substituent selected from bromo, iodo, C₁-C₆ alkoxy, 13 OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, 13 NO₂, —SO(C₁-C₄ alkyl), —SO₂(C₁-C₄ alkyl), —SO₂NH(C₁-C₄ alkyl) and —SO₂N(C₁-C₄alkyl)(C₁-C₂ alkyl);
—NR¹R² or CR¹R²R¹⁰ may form a saturated 3 to 8 membered carbocyclic ring which may optionally contain from one to three double bonds and wherein one or two of the ring carbon atoms of such 5 to 8 membered rings may optionally and independently be replaced by an oxygen or sulfur atom or by NZ³ wherein Z³ is hydrogen, C₁-C₄ alkyl, benzyl or C₁-C₄ alkanoyl;
R³ is hydrogen, C₁-C₄ alkyl, —O(C₁-C₄ alkyl), chloro, fluoro, bromo, iodo, (C₁-C₂ alkylene)-O—(C₁-C₂ alkyl), (C₁-C₂ alkylene)-OH, or —S(C₁-C₄ alkyl);
each R⁴ is, independently, hydrogen, (C₁-C₆ alkyl), fluoro, chloro, bromo, iodo, hydroxy, cyano, amino, (C₁-C₂ alkylene)-OH, CF₃, CH₂SCH₃, nitro, —O(C₁-C₄ alkyl), —N(C₁-C₄ alkyl)(C₁-C₂ alkyl), —S(C₁-C₄ alkyl), —CO(C₁-C₄ alkyl), —C(═O)H or —C(═O)O(C₁-C₄alkyl);
R⁶ is hydrogen, methyl or ethyl;
R⁸ is hydrogen or C₁-C₄ alkyl;
R⁵ is phenyl, pyridyl, pyrazinyl, pyrimidyl, pyridazinyl and wherein each of the foregoing R⁵ groups is substituted with from one to four substituents R¹³ wherein one to three of said substituents may be selected, independently, from fluoro, chloro, C₁-C₆ alkyl and —O(C₁-C₆ alkyl) and one of said substituents may be selected from bromo, iodo, formyl, OH, (C₁-C₄ alkylene)-OH, (C₁-C₄alkylene)-O—(C₁-C₂ alkyl), —CN, —CF₃, —NO₂, —NH₂, —NH(C₁-C₄ alkyl), —N(C₁-C₂ alkyl)(C₁-C₆ alkyl), —OCO(C₁-C₄ alkyl), (C₁-C₄ alkylene)-O—(C₁-C₄ alkyl), —S(C₁-C₆ alkyl), (C₁-C₄ alkylene)-S—(C₁-C₄ alkyl), —(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄alkyl moieties in the alkyl) and —SO₂(C₁-C₆ alkyl), and wherein each of the C₁-C₄ alkyl and C₁-C₆ alkyl moieties in the foregoing R⁵ groups may optionally have one or two double bonds;
R⁷ is hydrogen, C₁-C₄ alkyl, halo (e.g., chloro, fluoro, iodo or bromo), hydroxy, —O(C₁-C₄ alkyl), —C(═O)(C₁-C₄ alkyl), —C(═O)O(C₁-C₄ alkyl), —OCF₃, —CF₃, —CH₂OH or —CH₂O(C₁-C₂ alkyl);
R¹⁰ is hydrogen, hydroxy, methoxy or fluoro;
R¹¹ is hydrogen or C₁-C₄ alkyl; and

with the proviso that: a) when both J and K are carbons and D is CR⁴ and E is nitrogen, then G can not be nitrogen; (b) when both J and K are carbons and D and G are nitrogens, then E can not be CR⁴ or C═O or C═S; (c) when both J and K are carbons and D and E are carbons, then G can not be nitrogen; (d) when G is carbon, it must be double banded to E; and (e) in the ring containing J, K, D, E and G, there can not be two double bonds adjacent to each other;
and the pharmaceutically acceptable salts of such compounds.

VIII. Other useful CRF antagonists are of the following formula, disclosed in WO 98/08846: 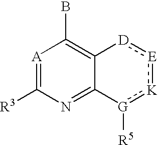
wherein the dashed lines represent optional double bonds;
A is nitrogen or CR;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or —COR²;
G is nitrogen or CR⁴ and is single bonded to all atoms to which it is attached, or G is carbon and is double bonded to K;
K is nitrogen or CR⁶ when double bonded to G or E, or K is oxygen, sulfur, C═O, C═S, CR⁶R¹² or NR⁸ when single bonded to both adjacent ring atoms, or K is a two atom spacer, wherein one of the two ring atoms of the spacer is oxygen, nitrogen, sulfur, C═O, C═S, CR⁶ R¹², NR⁶ or CR⁶, and the other is CR⁶ R¹² or CR⁹;
D and E are each, independently, C═O, C═S, sulfur, oxygen, CR⁴R⁶ or NR⁸ when single bonded to both adjacent ring atoms, or nitrogen or CR⁴ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;
R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur atom or by NZ wherein Z is hydrogen, C₁-C₄ alkyl or benzyl, and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ² wherein Z² is hydrogen, benzyl or C₁-C₄ alkyl;
R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);
each R⁸, R⁹ and R¹² is selected, independently, from hydrogen and C₁-C₂ alkyl;
each R⁴ and R⁶ that is attached to a carbon atom is selected, independently, from hydrogen and C₁-C₆ alkyl, fluoro, chloro, bromo, iodo, hydroxy, hydroxy (C₁-C₂ alkyl), trifluoromethyl, cyano, amino, nitro, —O(C₁-C₄ alkyl), —N(C₁-C₄ alkyl)(C₁-C₂ alkyl), —CH₂SCH₃, —S(C₁-C₄ alkyl), —CO(C₁-C₄ alkyl), —C(═O)H or —C(═O)O(C₁-C₄ alkyl), wherein each of the C₁-C₂ alkyl moieties in the foregoing R⁴ and R⁵ groups may optionally contain one double or triple bond; and R⁶, when attached to a nitrogen atom, is selected from hydrogen and C₁-C₄ alkyl;
R⁵ is substituted phenyl, naphthyl, pyridyl or pyrimidyl, wherein each of the foregoing R⁵ groups is substituted with from two to four substituents R¹³, wherein up to three of said substituents may be selected, independently, from chloro, C₁-C₆ alkyl, —O(C₁-C₆ alkyl) and —(C₁-C₆ alkylene)O(C₁-C₆alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, 13 SO₂NH(C₁-C₄ alkyl), —SO₂N(C₁-C₂ alkyl)(C₁-C₄ alkyl), —SO₂NH₂, —NHSO₂(C₁-C₄ alkyl), (C₀-C₁alkylene)-S—(C₁-C₂alkyl), —(CO-C₁alkylene)-SO—(C₁-C₂alkyl), —(C₀-C₁alkylene) —SO₂—(C₁-C₂alkyl) and —(C₁-C₄alkylene)-OH, and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo (e.g., chloro, fluoro, iodo or bromo), hydroxy, methoxy, —C(═O)(C₁-C₂ alkyl), —C(═O)O(C₁-C₂ alkyl), hydroxymethyl, trifluoromethyl or formyl;
R¹⁰ is hydrogen, hydroxy, methoxy or fluoro; and
R¹¹ is hydrogen or C₁-C₄ alkyl;
with the proviso that in the ring containing D, E, K and G of formula 1, there can not be two double bonds adjacent to each other;
and the pharmaceutically acceptable salt of such compound.

IX. The CRF antagonist may also be of the following formula, disclosed in WO 95/10506: 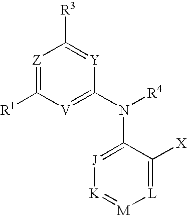
or a pharmaceutically, acceptable salt or prodrug thereof, wherein Y is CR^{3a}, N, or CR²⁹;
when Y is CR^{3a} or N:
R¹ is independently selected at each occurrence from the group consisting of C₁-C₄ alkyl, C₂-C₄ alkenyl, C₂-C₄ alkynyl, halogen, C₁-C₂ haloalkyl, NR⁶R⁷, OR⁸, and S(O)ₙR⁸; R³ is C₁-C₄ alkyl, aryl, C₃-C₆ cycloalkyl, C₁-C₂ haloalkyl, halogen, nitro, NR⁵R⁷, OR⁸, S(O)ₙR⁸ C(═O)R⁹, C(═O)NR⁶R⁷, C(═S)NR⁶R⁷, —(CHR¹⁶)ₖNR⁶R⁷, (CH₂)ₖOR⁸, C(═O)NR¹⁰CH(R¹¹)CO₂R¹², —C(OH)(R²⁵)(R^{25a}), —(CH₂)ₚS(O)ₙ-alkyl, —(CHR¹⁶)R²⁵, —C(CN)(R²⁵)(R¹⁶) provided that R²⁵ is not —NH— containing rings, —C(═O)R²⁵, —CH(CO₂R¹⁶)₂, NR¹⁰C(═O)CH(R¹¹)NR¹⁰R¹², NR¹⁰CH(R¹)CO₂R¹²; substituted C₁-C₄ alkyl, substituted C₂-C₄ alkenyl, substituted C₂-C₄ alkynyl, substituted C₁-C₄ alkoxy, aryl-(substituted C₁-C₄) alkyl, aryl-(substituted C₁-C₄) alkoxy, substituted C₃-C₆ cycloalkyl, amino-(substituted C₁-C₄)alkyl, substituted C₁-C₄ alkylamino, where substitution by R²⁷ can occur on any carbon containing substituent; 2-pyridinyl, imidazolyl, 3-pyridinyl, 4-pyridinyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-pheno-thiazinyl, 4-pyrazinyl, azetidinyl, phenyl, 1H-indazolyl, 2-pyrrolidonyl, 2H,6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azocinyl, azepinyl, benzofuranyl, benzothiophenyl, carbazolyl, chromanyl, chromenyl, cinnolinyl, decahydroquinolinyl, furazanyl, imidazolidinyl, indolinyl, indolizinyl, indolyl, isobenzofuranyl, isochromanyl, isoindolinyl, isoindolyl, isoquinolinyl, benzimidazolyl, isothiazolyl, isoxazolyl, morpholinyl, naphthyridinyl, octahydroisoquinolinyl, oxazolidinyl, oxazolyl, phenanthridinyl, phenanthrolinyl, phenazinyl, phenoxathiinyl, phenoxazinyl, phthalazinyl, piperazinyl, piperidinyl, pteridinyl, purinyl, pyranyl, pyrazolidinyl, pyrazolinyl, pyrazolyl, pyridazinyl, pyrimidinyl, pyrrolidinyl, pyrrolinyl, pyrrolyl, quinazolinyl, quinolinyl, quinoxalinyl, quinuclidinyl, β-carbolinyl, tetrahydrofuranyl, tetrahydroisoquinolinyl, tetrahydroquinolinyl, tetrazolyl, thianthrenyl, thiazolyl, thiophenyl, triazinyl, xanthenyl; or 1-tetrahydroquinolinyl or 2-tetrahydroisoquinolinyl either of which can be substituted with 0-3 groups chosen from keto and C₁-C₄ alkyl; J, K, and L are independently selected at each occurrence from the group of N, CH, and CX′;
M is CR⁵ or N;
V is CR ^{1a} or N;
Z is CR²or N;
R^{1a}, R², and R^{3a} are independently selected at each occurrence from the group consisting of hydrogen, halo, halomethyl, C₁-C₃ alkyl, and cyano;
R⁴ is (CH₂)ₘOR¹⁶, C₁-C₄ alkyl, allyl, propargyl, (CH₂)ₘR¹³, or —(CH₂)ₘOC(O)R¹⁶;
X is halogen, aryl, heteroaryl, S(O)₂R⁸, SR⁸, halomethyl, —(CH₂)ₚOR⁸, cyano, —(CHR¹⁶)ₚNR¹⁴R¹⁵, —C(═O)R⁸, C₁-C₆ alkyl, C₄-C₁₀ cycloalkylalkyl, C₁l-C₁₀alkenyl, C₂-C₁₀alkynyl C₂-C₁₀alkoxy, aryl-(C₂-C₁₀)-alkyl, C₃-C₆cycloalkyl, aryl-(C₁-C₁₀)-alkoxy, nitro, thio-(C₁-C₁₀)-alkyl, —C(═NOR¹⁶)—C₁-C₄-alkyl, —C(═NOR¹⁶)H, or —C(═O)NR¹⁴R¹⁵, where substitution by R¹⁸ can occur on any carbon containing substituents;
X′ is independently selected at each occurrence from the group consisting of hydrogen, halogen, aryl, heteroaryl, S(O)ₙR⁸, halomethyl, —(CHR¹⁶)ₚOR⁸, cyano, —(CHR¹⁶)ₚNR¹⁴R¹⁵, C(═O)R⁸, C₁-C₆ alkyl, C₂-C₁₀alkenyl, C₂-C₁₀alkynyl, C₁-C₁₀alkoxy, aryl-(C₁-C₁₀)-alkyl, C₃-C₆cycloalkyl, aryl-(C₁-C₁₀)-alkoxy, nitro, thio-(C₁-C₁₀)-alkyl, —C(═NOR¹⁶)—C₁-C₄-alkyl, —C(═NOR¹⁶)H, and —C(═O)NR¹⁴R¹⁵, where substitution by R¹⁶ can occur on any carbon containing substituents;
R⁵ is halo, —C(═NOR¹⁶)—C₁-C₄-alkyl, C₁-C₄alkyl, C₁-C₃ haloalkyl, —(CHR¹⁶)ₚOR⁸, —(CHR¹⁶)ₚS(O)ₙR⁸, —(CHR⁶)ₚNR¹⁴R¹⁵, C₃-C₆ cycloalkyl, C₂-C₁₀alkenyl, C₂-C₁₀alkynyl, aryl-(C₂-C₁₀)-alkyl, aryl-(C₁-C₁₀)-alkoxy, cyano, C₃-C₆ cycloalkoxy, nitro, amino-(C₂-C₁₀)-alkyl, thio-(C₂-C₁₀)-alkyl, SOₙ(R⁸), C(═O)R⁸—C(═NOR¹⁶)H, or —C(═O)NR¹⁴R¹⁵, where substitution by R¹⁸ can occur on any carbon containing substituents;
R⁶ and R⁷ are independently selected at each occurrence from the group consisting of hydrogen, C₁-C₆ alkyl, C₃-C₁₀ cycloalkyl, C₁-C₆ alkoxy, (C₄-C₁₂)-cycloalkylalkyl, —(CH₂)ₖR¹³, (CHR¹⁶)ₚOR⁸, —(C₁-C₆alkyl)-aryl, heteroaryl, —S(O)₂-aryl or —(C₁-C₆alkyl)-heteroaryl or aryl, wherein the aryl or heteroaryl groups are optionally substituted with 1-3 groups selected from the group consisting of hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), cyano, and S(O)₂—(C₁-C₆-alkyl); or can be taken together to form —(CH₂)ₚ(CH₂)ᵣ—, optionally substituted with 0-3 R¹⁷; or, when considered with the commonly attached nitrogen, can be taken together to form a heterocycle, said heterocycle being substituted on carbon with 1-3 groups consisting of hydrogen, C₁-C₆ alkyl, hydroxy, or C₁-C₆ alkoxy;
A is CH₂, O, NR²⁵, C(═O), S(O)ₙ, N(C(═O)R¹⁷), N(R¹⁹), C(H)(NR¹⁴R¹⁵), C(H)(OR²⁰), C(H)(C(═O)R²¹), or N(S(O)ₙR²¹);
R⁸ is independently selected at each occurrence from the group consisting of hydrogen; C₁-C₆ alkyl; —(C₄-C₁₂) cycloalkylalkyl; (CH₂)ₜR²²; C₃-C₁₀ cycloalkyl; —NR⁶R⁷; aryl; heteroaryl; —NR¹⁶(CH₂)ᵣR⁶R⁷; —(CH₂)ₖR^{25;} and (CH₂)ₜheteroaryl or (CH₂)ₜaryl, either of which can optionally be substituted with 1-3 groups selected from the group consisting of hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), cyano, and S(O)₂(C₁-C₆-alkyl);
R⁹ is independently selected at each occurrence from R¹⁰, hydroxy, C₁-C₄ alkoxy, C₃-C₆ cycloalkyl, C₂-C₄ alkenyl, aryl substituted with 0-3 R¹⁸, and —(C₁-C₆ alkyl)-aryl substituted with 0-3 R¹⁸;
R¹⁰, R¹⁶, R²⁴, and R² are independently selected at each occurrence from hydrogen or C₁-C₄ alkyl;
R¹¹ is C₁-C₄ alkyl substituted with 0-3 groups chosen from the following: keto, amino, sulfhydryl, hydroxyl, guanidinyl, p-hydroxyphenyl, imidazolyl, phenyl, indolyl, and indolinyl, or, when taken together with an adjacent R¹⁰, are (CH₂)ₜ;
R¹² is hydrogen or an appropriate amine protecting group for nitrogen or an appropriate carboxylic acid protecting group for carboxyl;
R¹³ is independently selected at each occurrence from the group consisting of CN, OR¹⁹, SR¹⁹, and C₃-C₆ cycloalkyl;
R¹⁴ and R¹⁵ are independently selected at each occurrence from the group consisting of hydrogen, C₄-C₁₀, cycloalkyl-alkyl, and R₁₉;

R¹⁷ is independently selected at each occurrence from the group consisting of R¹⁰, C₁-C₄ alkoxy, halo, OR²³, SR²³, NR²³R²⁴, and (C₁-C₆) alkyl (C₁-C₄) alkoxy;
R₁₈ is independently selected at each occurrence from the group consisting of R¹⁰, hydroxy, halogen, C₁-C₂ haloalkyl, C₁-C₄ alkoxy, C(═O)R²⁴, and cyano;
R¹⁹ is independently selected at each occurrence from the group consisting of C₁-C₆ alkyl, C₃-C₆ cycloalkyl, (CH₂)_{w}R²², and aryl substituted with 0-3 R¹⁸;
R²⁰ is independently selected at each occurrence from the group consisting of R¹⁰, C(═O)R³¹, and C₂-C₄ alkenyl;
R²¹ is independently selected at each occurrence from the group consisting of R¹⁰, C₁-C₄ alkoxy, NR²³R²⁴, and hydroxyl;
R²² is independently selected at each occurrence from the group consisting of cyano, OR²⁴, SR²⁴, NR²³R²⁴,C₁-C₆ alkyl, C₃-C₆ cycloalkyl, —S(O)ₙR³¹, and —C(═O)R²⁵;
R²⁵, which can be optionally substituted with 0-3 R¹⁷, is independently selected at each occurrence from the group consisting of phenyl, pyrazolyl, imidazolyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-pheno-thiazinyl, 4-pyrazinyl, azetidinyl, 1H-indazolyl, 2-pyrrolidonyl, 2H,6H-1,5,2-dithiazinyl, 2H-pyrrolyl, 3H-indolyl, 4-piperidonyl 4aH-carbazolyl, 4H-quinolizinyl, 6H-1,2,5-thiadiazinyl, acridinyl, azocinyl, azepinyl, benzofuranyl, benzothiophenyl, carbazolyl, chromanyl, chromenyl, cinnolinyl, decahydroquinolinyl, furazanyl, indolinyl, indolizinyl, indolyl, isobenzofuranyl, isochromanyl, isoindolinyl, isoindolyl, isoquinolinyl benzimidazolyl, isothiazolyl, isoxazolyl, morpholinyl, naphthyridinyl, octahydroisoquinolinyl, oxazolidinyl, oxazolyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazolidinyl, pyridazinyl, pyridyl, pyrimidinyl, pyrrolidinyl, pyrrolinyl, pyrrolyl, quinazolinyl, quinolinyl, quinoxalinyl, quinuclidinyl, B-carbolinyl, tetrahydrofuranyl, tetrazolyl, thianthrenyl, thiazolyl, thiophenyl, triazinyl, xanthenyl; and 1-tetrahydroquinolinyl or 2-tetrahydroisoquinolinyl either of which can be substituted with 0-3 groups chosen from keto and C₁-C₄ alkyl;
R^{25a}, which can be optionally substituted with 0-3 R¹⁷, is independently selected at each occurrence from the group consisting of H and R²⁵;
R²⁷ is independently selected at each occurrence from the group consisting of C₁-C₃ alkyl, C₂-C₄ alkenyl, C₂-C₄ alkynyl, C₂-C₄ alkoxy, aryl, nitro, cyano, halogen, aryloxy, and heterocycle optionally linked through 0;
R³¹ is independently selected at each occurrence from the group consisting of C₁-C₄ alkyl, C₃-C₇ cycloalkyl, C₄-C₁₀ cycloalkyl-alkyl, and aryl-(C₁-C₄) alkyl;
k, m, and r are independently selected at each occurrence from 1-4;
n is independently, selected at each occurrence from 0-2,
p, q, and z are independently selected at each occurrence from 0-3;
t and w are independently selected at each occurrence from 1-6,
provided that when J is CX′ and K and L are both CH, and M is CR⁵, then
(A) when V and Y are N and Z is CH and R¹ and R³ are methyl,
   (1) and R⁴ is methyl, then
      (a) R⁵ can not be methyl when X is OH and X′ is H;
      (b) R⁵ can not be —NHCH₃, or -N(CH₃)₂ when X and X′ are —OCH₃; and
      (c) R⁵ can not be —N(CH₃)₂ when X and X′ are —OCH₂CH₃;
   (2) and R⁴ is ethyl, then
      (a) R⁵ can not be methylamine when X and X′ are —OCH₃;
      (b) R⁵ can not be OH when X is Br and X′ is OH; and
      (c) R⁵ can not be —CH₂OH or —CH₂N(CH₃)₂ when X is —SCH₃ and X′ is H;
(B) when V and Y are N, Z is CH, R⁴ is ethyl, R⁵ is iso-propyl, X is Br, X′ is H, and
   (1) R¹ is CH₃, then
      (a) R³ can not be OH, piperazin-1-yl, —CH₂,-piperidin-1-yl, —CH₂—(N-4-methylpiperazin-1-yl), —C(O)NH-phenyl, —CO₂H, —CH₂O-(4-pyridyl), —C(O)NH₂, 2-indolyl, —CH₂O-(4-carboxyphenyl), —N(CH₂CH₃)(2-bromo-4-isopropylphenyl);
   (2) R² is —CH₂CH₂CH₃ then R³ can not be —CH₂CH₂CH₃
(C) when V, Y and Z are N, R⁴ is ethyl, and
   (1) R⁵ is iso-propyl, X is bromo, and X′ is H, then
      (a) R³ can not be OH or —OCH₂CN when R¹ is CH₃ and
      (b) R³can not be —N(CH₃)₂ when R¹ is —N(CH₃)₂;
   (2) R⁵ is —OCH₃, X is —OCH₃, and X′ is H, then R³ and R¹ can not both be chloro; further provided that when J, K, and L are all CH and M is CR⁵, then
(D) at least one of V, Y, and Z must be N;
(E) when V is CR^{1a}, Z and Y can not both be N;
(F) when Y is CR^{3a}, Z and V can not both be N;
(G) when Z is CR², V and Y must both be N;
(H) Z can be N only when both V and Y are N or when V is CR^{1a} and Y is CR^{3a};
(I) when V and Y are N, Z is CR², and R² is H or C₁-C₃ alkyl, and R⁴ is C₁-C₃ alkyl, R³ can not be 2-pyridinyl, indolyl, indolinyl, imidazolyl, 3-pyridinyl, 4-pyridinyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-phenothiazinyl, or 4-pyrazinyl;
(J) when V and Y are N; Z is CR²; R² is H or C₁-C₃ alkyl; R⁴ is C₁-C₄ alkyl, R⁵, X, and/or X′ are OH, halo, CF₃, C₁-C₄ alkyl, C₁-C₄ alkoxy, C₁-C₄ alkylthio, cyano, amino, carbamoyl, or C₁-C₄ alkanoyl; and R¹ is C₁-C₄ alkyl, then R⁴ can not be —NH(substituted phenyl) or —N(C₁-C₄ alkyl) (substituted phenyl);
and wherein, when Y is CR²⁹:
J, K, L, M, Z, A, k, m, n, p, q, r, t, w, R³, R¹⁰, R¹¹, R¹², R¹³, R¹⁶, R¹⁸, R¹⁹, R²¹, R²³ , R²⁴, R²⁵, and R²⁷ are as defined above and R^{25a}, in addition to being as defined above, can also be C₁-C₄ alkyl, but
V is N;
R¹ is C₁-C₂ alkyl, C₂-C₄ alkenyl, C₂-C₄ alkynyl, C₂-C₄ alkoxy, halogen, amino, methylamino, dimethylamino, aminomethyl, or N-methylaminomethyl;
R² is independently selected at each occurrence from the group consisting of hydrogen, halo, C₁-C₃, alkyl, nitro, amino, and —CO₂R¹⁰;

R₄ is taken together with R²⁹ to form a 5-membered ring and is —C(R²⁶)═or —N═ when R²⁹ is —C(R³⁰)═ or —N═, or —CH(R²⁶)— when R²⁹ is —CH(R³⁰)—;
X is Cl, Br, I, S(O)nR⁸, OR⁸, halomethyl, —(CHR¹⁶)ₚOR⁸, cyano, —(CHR¹⁶)ₚNR¹⁴R¹⁵, C(═O)R⁸, C₁-C₆ alkyl, C₂-C₁₀ alkenyl, C₂-C₁₀ alkynyl, C₁-C₁₀, alkoxy, aryl-(C₁-C₁₀)-alkyl, C₃-C₆ cycloalkyl, aryl-(C₁-C₁₀)-alkoxy, nitro, thio-(C₁-C₁₀)-alkyl, —C(═NOR¹⁶)—C₁-C₄-alkyl, —C(═NOR¹⁶)H, or C(═O)NR¹⁴R¹⁵ where substitution by R¹⁸ can occur on any carbon containing substituents;
X′ is hydrogen, Cl, Br, I, S(O)ₙR⁸, —(CHR¹⁶)ₚOR⁸, halomethyl, cyano, —(CHR¹⁶)ₚNR¹⁴R¹⁵, C(═O)R⁸, C₁-C₆ alkyl, C₂-C₁₀alkenyl, C₂-C₁₀, alkynyl, C₁-C₁₀alkoxy, aryl-(C₁-C₁₀)-alkyl, C₃-C₆ cycloalkyl, aryl-(C₂-C₁₀)-alkoxy, nitro, thio-(C₂-C₁₀)-alkyl, —C(═NOR¹⁶)—C₁-C₄-alkyl, —C(═NOR¹⁶)H, or C(═O)NR⁸R¹⁵ where substitution by R¹⁸ can occur on any carbon containing substituents;
R⁵ is halo, —C(═NOR¹⁶)—C₁-C₄-alkyl, C₁-C₆ alkyl, C₁-C₃ haloalkyl, C₁-C₆ alkoxy, (CHR¹⁶)ₚOR⁵, (CHR¹⁶)ₚS(O)ₙR⁸, (CHR¹⁶)ₚNR¹⁴R¹⁵, C₃-C₆ cycloalkyl, C₂-C₁₀ alkenyl, C₂-C₁₀ alkynyl, aryl-(C₂-C₁₀)-alkyl, aryl-(C₁-C₁₀)-alkoxy, cyano, C₃-C₆ cycloalkoxy, nitro, amino-(C₁-C₁₀)-alkyl, thio-(C₁-C₁₀)-alkyl, SOₙ(R⁸), C(═O)R⁸, —C(═NOR¹⁶)H, or C(═O)NR⁸R¹⁵ where substitution by R¹⁸ can occur on any carbon containing substituents;
R⁶ and R⁷ are independently selected at each occurrence from the group consisting of hydrogen, C₁-C₆ alkyl, C₃-C₁₀ cycloalkyl, —(CH₂)ₖR¹³, (C₄-C₁₂)-cycloalkylalkyl, C₁-C₆ alkoxy, —(C₁-C₆ alkyl)-aryl, heteroaryl, aryl, —S(O)_{z}-aryl or —(C₁-C₆ alkyl)-heteroaryl or aryl wherein the aryl or heteroaryl groups are optionally substituted with 1-3 groups selected from hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), and cyano; or can be taken together to form —(CH₂)_{q}A(CH₂)ᵣ—, optionally substituted with 0-3 R¹⁷; or, when considered with the commonly attached nitrogen, can be taken together to form a heterocycle, said heterocycle being substituted on carbon with 1-3 groups consisting of hydrogen, C₁-C₆ alkyl, hydroxy, or C₁-C₆ alkoxy;
R⁸ is independently selected at each occurrence from the group consisting of hydrogen, C₁-C₆ alkyl, —(C₄-C₁₂) cycloalkylalkyl, (CH₂)ₜR²², C₃-C₁₀ cycloalkyl, —(C₁-C₆ alkyl)-aryl, heteroaryl, —NR¹⁶, —N(CH₂)ₙNR⁶R⁷; —(CH₂)ₖR²⁵, —(C₁-C₆ alkyl)-heteroaryl or aryl optionally substituted with 1-3 groups selected from hydrogen, halogen, C₁-C₆ alkyl, C₁-C₆ alkoxy, amino, NHC(═O)(C₁-C₆ alkyl), NH(C₁-C₆ alkyl), N(C₁-C₆alkyl)₂, nitro, carboxy, CO₂(C₁-C₆ alkyl), and cyano;

R⁹ is independently selected at each occurrence from R¹⁰, hydroxy, C₁-C₄ alkoxy, C₃-C₆ cycloalkyl, C₂-C₄ alkenyl, and aryl substituted with 0-3 R^{18;}
R¹⁴ and R¹⁵ are independently selected at each occurrence from the group consisting of hydrogen, C₁-C₆ alkyl, C₃-C₆ cycloalkyl, (CH₂)ₜR²², and aryl substituted with 0-3 R¹⁸;
R¹⁷ is independently selected at each occurrence from the group consisting of R¹⁰, C₁-C₄ alkoxy, halo, OR²³, SR²³, and NR²³R²⁴;
R²⁰ is independently selected at each occurrence from the group consisting of R¹⁰ and C(═O)R³¹;
R²² is independently selected at each occurrence from the group consisting of cyano, OR²⁴, SR²⁴, NR²³R²⁴, C₃-C₆ cycloalkyl, —S(O)ₙR³¹, and —C(═O)R²⁵;
R²⁶ is hydrogen or halogen;
R²⁸ is C₁-C₂, alkyl, C₂-C₄ alkenyl, C₂-C₄ alkynyl, hydrogen, C₁-C₂ alkoxy, halogen, or C₂-C₄ alkylamino;
R²⁹ is taken together with R⁴ to form a five membered ring and is: —CH(R³⁰)— when R⁴ is —CH(R²⁸)—, —C(R³⁰)═ or—N═ when R⁴ is —C(R²⁸)═ or —N═;

R³⁰ is hydrogen, cyano, C₁-C₂ alkyl, C₁-C₂ alkoxy, halogen, C₁-C₂ alkenyl, nitro, amido, carboxy, or amino;
R³¹ is C₁-C₄ alkyl, C₃-C₇ cycloalkyl, or aryl-(C₁-C₄) alkyl; provided that when J, K, and L are all CH, M is CR⁵, Z is CH, R³ is CH₃, R²⁸ is H, R⁵ is isopropyl, X is Br, X′ is H, and R¹ is CH₃, then R³⁰ can not be H, —CO₂H, or —CH₂NH₂; and further provided that when J, K and L are all CH; M is CR⁵; Z is N; and
(A) R² is —C(R³⁰)═; then one of R²⁸ or R³⁰ is hydrogen;
(B) R²⁹ is N; then R³ is not halo, NH₂, NO₂, CF₃, CO₂H, CO₂-alkyl, alkyl, acyl, alkoxy, OH, or —(CH₂)ₘOalkyl;
(C) R²⁹ is N; then R²⁸ is not methyl if X or X′ are bromo or methyl and R⁵ is nitro; or
(D) R²⁹ is N; and R¹ is CH₃; and R³ is amino; then R⁵ is not halogen or methyl.

Preferred compounds of this group include those wherein:
i) V is N, R¹ is methyl; and R³ is aryl, NR⁶R⁷, or OR⁸;
ii) V is N, R¹ is methyl; R³ is aryl, NR⁶R⁷, or OR⁸; and R⁴ is methyl or ethyl;
iii) V is N, R¹ is methyl; R³ is aryl, NR⁶R⁷, or OR⁸; R⁴ is methyl or ethyl; and X is O(C₁-C₄ alkyl), Br, or C₁-C₄ alkyl;
iv) V is N, R¹ is methyl; R³ is aryl, NR⁶R⁷, or OR⁸; R⁴ is methyl, ethyl; X is OMe, Br, or (C₁-C₄ alkyl), M is C₁-C₄ alkyl, Br, Cl, or O(C₁-C₄ alkyl); and
v) V is N, R¹ is methyl; R³ is aryl, NR⁶R⁷, OR⁸; or R⁴ is methyl, ethyl; X is OMe, Br, or C₁-C₄ alkyl, M is C₁-C₄ alkyl, Br, Cl, or O(C₁-C₄ alkyl); and L is CH, or N.

X. The invention also encompasses use of aminothiazole derivatives of the following formula, disclosed in WO 97/00868: 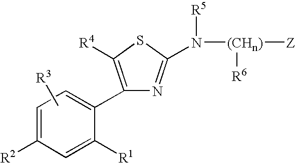
wherein each of R¹ and R² is independently a halogen atom; a C₁₋C₅ hydroxyalkyl radical; C₁-C₅ alkyl; C₇-C₁₀ aralkyl; C₁₋C₅ alkoxy; trifluoromethyl; nitro; nitrile; a group —SR where R is hydrogen, a C₁-C₅ alkyl radical or a C₇-C₁₀ aralkyl radical; a group S—CO—R where R is a C₁-C₅ alkyl radical or aralkyl in which the aryl portion is C₆-C₈ and the alkyl portion is C₁-C₄; a group —COOR′ where R′ is hydrogen or C₁-C₅ alkyl; a group —CONR′R″ where R′ and R″ are as defined above for R′; a group —NR′R″ where R′ and R″ are as previously defined for R′; a group —CONRaRb or NRaRb, where Ra and Rb, taken together with the nitrogen atom to which they are attached, form a 5- to 7-membered heterocyclic ring; or a group —NHCO—NR′R″, where R′ and R″ are as defined above for R′; R³ is hydrogen or as defined for R¹ and R² is a hydrogen atom; C₁₋₅ alkyl; halogen; a hydroxymethyl group; or a formyl group; R⁵ is C₁-C₅ alkyl; a C₃-C₇ cycloalkyl group; a cycloalkylalkyl group in which the cycloalkyl portion is C₃-C₇ and the alkyl portion is C₁-C₅; or C₅-C₆ alkenyl; n is 0 or 1; R⁶ is C₁₋₅ alkyl; alkoxyalkyl in which the alkyl portions are C₁-C₅; C₃-C₇ cycloalkyl; a cycloalkylalkyl group in which the cycloalkyl portion is C₃-C₇ and the alkyl portion is C₁-C₅; a cycloalkyloxyalkyl radical in which the cycloalkyl is C₃-C₇ and the alkyl is C₁-C₄; a hydroxyalkyloxyalkyl radical in which the alkyls are C₂-C₁₀; or an alkoxyalkyloxyalkyl radical in which the alkyls are C₃-C₁₂; and Z is an optionally substituted bi- or tricyclic aromatic or heteroaromatic group; and stereoisomers and/or addition salts thereof.

XI. CRF antagonists of the following formula, disclosed in WO 97/29109, may also be employed: 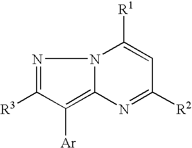
including the stereoisomers and the pharmaceutically acceptable acid addition salt
forms thereof, wherein
R¹ is NR⁴R⁵or OR⁵;
R² is C₁-C₆alkyl, C₁-C₆alkyloxy or C₁-C₆alkylthio,
R³ is hydrogen, C₁-C₆alkyl, C₁-C₆alkylsulfonyl, C₁-C₆alkylsulfoxy or C₁-C₆alkylthio;
R⁴ is hydrogen, C₁-C₆alkyl, mono- or di(C₃-C₆cyloalkylmethyl, C₃-C₆cyloalkyl, C₃-C₆alkenyl, hydroxyC₁-C₆alkyl, C₁-C₆akylcarbonyloxyC₁-C₆alkyl or C₁-C₆alkyloxyC₁-C₆alkyl;

R⁵ is C₁-C₈alkyl, mono- or di(C₃-C₆cycloalkyl)methyl, Ar¹CH₂, C₃-C₆alkenyl, C₁-C₆alkyloxyC₁-C₆alkyl, hydroxyC₁-C₆alkyl, thienylmethyl, furanylmethyl, C₁-C₆alkylthioC₁-C₆alkyl, morpholinyl, mono- or di(C₁-C₆alkyl)aminoC₁alkyl, di(C₁-C₆alkyl)amino, C₁-C₆alkylcarbonylC₁-C₆alkyl, C₁-C₆alkyl substituted with imidazolyl; or a radical of formula —Alk-O—CO—Ar¹;
or R⁴ and R⁵ taken together with the nitrogen atom to which they are attached may form a pyrrolidinyl, piperidinyl, homopiperidinyl or morpholinyl group, optionally substituted with C₁-C₆alkyl or C₁-C₆alkyloxyC₁-C₆alkyl; and
Ar is phenyl; phenyl substituted with 1, 2 or 3 substituents independently selected from halo, C₁-C₆alkyl, trifluoromethyl, hydroxy, cyano, C₁-C₆alkyloxy, benzyloxy, C₁-C₆alkylthio, nitro, amino and mono- or di(C₁-C₆alkyl)amino; pyridinyl; pyridinyl substituted with I ˜2 or 3 substituents independently selected from halo, C₁-C₆alkyl, trifluoromethyl, hydroxy, cyano, C₁-C₆alkyloxy, benzyloxy, C₁-C₆alkylthio, nitro, amino, mono- or di(C₁-C₆alkyl)amino and piperidinyl; and wherein said substituted phenyl may optionally be further substituted with one or more halogens;
Ar¹ is phenyl; phenyl substituted with 1, 2 or 3 substituents each independently selected from halo, C₁-C₆alkyl, C₁-C₆alkyloxy, di(C₁-C₆alkyl)aminoC₁-C₆alkyl, trifluoromethyl and C₁-C₆alkyl substituted with morpholinyl; or pyridinyl; and Alk is C₁-C₆alkanediyl; with the proviso that 5-methyl-3-phenyl-7-(phenylmethoxy)-pyrazolo[1,5-a]-pyrimidine and 2,5-dimethyl-7-(methylamino)-3-phenyl-pyrazolo[1,5-a]pyrimidine are not included.

Preferred compounds of this formula are those wherein R² is methyl; R³is hydrogen, or C₁-C₆ alkyl; and Ar is substituted phenyl or 3-pyridyl.

XII. CRF antagonists of the following formula, disclosed in WO 97/29110, may also be employed: 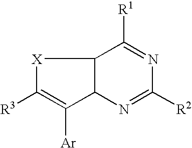
including the stereoisomers and the pharmaceutically acceptable acid addition salt
forms thereof, wherein
X is S. SO or SO₂;
R¹ is NR⁴R⁵ or OR⁵;
R² is C₁-C₆alkyl, C₁-C₆alkyloxy or C₁-C₆alkylthio;
R³is hydrogen, C₁-C₆alkyl, C₁-C₆alkylsulfonyl, C₁-C₆alkylsulfoxy or C₁-C₆alkylthio;
R⁴ is hydrogen, C₁alkyl, mono- or di(C₃-C₆cycloalkyl)methyl, C₃-C₆cycloalkyl, C₃-C₆alkenyl, hydroxyC₁-C₆alkyl, C₁-C₆alkylcarbonyloxyC₁-C₆alkyl or C₁-C₆alkyloxyC₁-C₆alkyl;
R⁵ is C₁-C₈alkyl, mono- or di(C₃-C₆cycloalkyl)methyl, Ar¹CH₂, C₃-C₆alkenyl, C₁-C₆alkyloxyC₁-C₆alkyl, hydroxyC₁-C₆alkyl, thienylmethyl, furanylmethyl, C₁-C₆alkylthioC₁-C₆alkyl, morpholinyl, mono- or di(C₁-C₆alkyl)aminoC₁-C₆alkyl, di(C₁-C₆alkyl)amino, C₁-C₆alkylcarbonylC₁-C₆alkyl, C₁-C₆alkyl substituted with imidazolyl; or a radical of formula —Alk-O—CO—Ar I; or R⁴ and R⁵ taken together with the nitrogen atom to which they are attached may form a pyrrolidinyl, piperidinyl, homopiperidinyl or morpholinyl group, optionally substituted with C₁-C₆alkyl or C₁-C₆alkyloxyC₁-C₆alkyl;
Ar is phenyl; phenyl substituted with 1, 2 or 3 substituents independently selected from halo, C₁-C₆alkyl, trifluoromethyl, hydroxy, cyano, C₁-C₆alkyloxy, benzyloxy, C₁-C₆alkylthio, nitro, amino and mono- or di(C₁-C₆alkyl)amino; pyridinyl; pyridinyl substituted with 1, 2 or 3 substituents independently selected from halo, C₁-C₆alkyl, trifluoromethyl, hydroxy, cyano, C₁-C₆alkyloxy, benzyloxy, C₁-C₆alkylthio, nitro, amino, mono- or di(C₁-C₆alkyl)amino and piperidinyl; and wherein said substituted phenyl may optionally be further substituted with one or more halogens;
Ar¹ is phenyl; phenyl substituted with 1, 2 or 3 substituents each independently selected from halo, C₁-C₆alkyl, C₁-C₆alkyloxy, di(C₁-C₆alkyl)aminoC,-C₆alkyl trifluoromethyl, and C₁-C₆alkyl substituted with morpholinyl; or pyridinyl; and
Alk is C₁-C₆alkanediyl.

Preferred compounds of this group include those wherein:
i) R² is methyl;
ii) R² is methyl; and Ar is substituted phenyl or 3-pyridyl;
iii) R² is methyl; R³ is methyl; and Ar is substituted phenyl or 3-pyridyl.

Specific CRF antagonists useful in the practice of the invention, include, without limitation, the following compounds:

14-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-( 1-ethyl-propyl)-amine;
(3,6-dimethyl-2-(4-chloro-2,6-dimethyl-phenoxy)-pyridin-4-yl)-(1-ethyl-propyl)-amine;
5-(1-ethyl-propoxy)-7-methyl-1-(2,6-dimethyl-4-cholorophenyl)-1-4-dihydro-2H-3-oxa-1,8-diazanaphthalene;
butyl-[2,5-dimethyl-7-(2,4,6-trimethylphenyl)-6,7-dihydro-5H-pyrrolo[2,3-d]pyrimidin4-yl]-ethyl-amino;
4-(butyl-ethylamino)-2,5-dimethyl-7-(2,4,6-trimethylphenyl)-5,7-dihydro-pyrrolo[2,3-d]pyrimidin-6-one;
4-(1-ethylpropoxy)-2,5-dimethyl-6-(2,4,6-trimethylphenoxy)-pyrimidine;
N-butyl-N-ethyl-2,5-dimethyl-NN-(2,4,6-trimethylphenyl)-pyrimidine-4,6-diamine;
[4-(1-ethyl-propoxy)-3,6-dimethyl-pyridin-2-yl]-(2,4,6-trimethylphenyl)-amine;
6-(ethyl-propyl-amino)-2,7-dimethyl-9-(2,4,6-trimethylphenyl)-7,9-dihydro-purin-8-one;
3-{(4-methyl-benzyl)-[3,6-dimethyl-1-(2,4,6-trimethylphenyl)-1 H-pyrazolo[3,4-d]pyrimidin-4-yl]-amino}-propan-1-ol;
diethyl-[6-methyl-3-methylsulfanyl-1-(2,4,6-trichlorophenyl)-1H-pyrazolo[3,4-d]pyrimidin-4-yl]-amine;
2butyl-[6-methyl-3-methylsulfanyl-1-(2,4,6-trichlorophenyl)-1H-pyrazolo[3,4-d]pyrimidin-4-yl]-amino}-ethanol;
dibutyl-[6-methyl-3-methylsulfanyl-1-(2,4,6-trichlorophenyl)-1H-pyrazolo[3,4-d]pyrimidin-4-yl}-amine;
butyl-ethyl-[6-methyl-3-methylsulfanyl-1-(2,4,6-trichlorophenyl)-1H-pyrazolo[3,4-d]pyrimidin-4-yl]-amine;
butyl-ethyl-[6-methyl-3-methylsulfonyl-1-(2,4,6-trichlorophenyl)-1H-pyrazolo[3,4-d]pyrimidin4-yl]-amine;
butyl-cyclopropylmethyl-[6-methyl-3-methylsulfanyl-1-(2,4,6-trichlorophenyl)-1H-pyrazolo[3,4-d]pyrimidin-4-yl]-amine;
di-1-propyl-[6-methyl-3-methylsulfanyl-1-(2,4,6-trichlorophenyl)-1H-pyrazolo[3,4-d]pyrimidin-4-yl]-amine;
diallyl-[6-methyl-3-methylsulfanyl-1-(2,4,6-trichlorophenyl)-1H-pyrazolo[3,4-d]pyrimidin-4-yl]-amine;
butyl-ethyl-[6-chloro-3-methylsulfanyl-1-(2,4,6-trichlorophenyl)-1H-pyrazolo[3,4-d]pyrimidin-4-yl]-amine;
butyl-ethyl-[6-methoxy-3-methylsulfanyl-1-(2,4,6-trichlorophenyl)-1H-pyrazolo[3,4-d]pyrimidin4-yl]-amine;
propyl-ethyl-[3,6-dimethyl-1-(2,4,6-trimethylphenyl)-1H-pyrazolo[3,4-d]pyrimidin-4-yl]-amine;
4-(1-ethyl-propyl)-6-methyl-3-methylsulfanyl-1-(2,4,6-trimethylphenyl)-1H-pyrazolo[3,4-d]pyrimidine;
n-butyl-ethyl-[2,5-dimethyl-7-(2,4,6-trimethylphenyl)-7H-pyrrolo[2,3-d]pyrimidin-4-yl]amine;
di-n-propyl-[2,5-dimethyl-7-(2,4,6-trimethylphenyl)-7H-pyrrolo[2,3-d]pyrimidin-4-yl]amine;
ethyl-n-propyl-[2,5-dimethyl-7-(2,4,6-trimethylphenyl)-7H-pyrrolo[2,3-d]pyrimidin-4-yl]amine;
diethyl-2,5-dimethyl-7-(2,4,6-trimethylphenyl)-7H-pyrrolo[2,3-d]pyrimidin-4-yl]amine;
n-butyl-ethyl-[2,5,6-trimethyl-7-(2,4,6-trimethylphenyl)-7H-pyrrolo[2,3-d]pyrimidin4-yl]amine;
2-{N-n-butyl-N-[2,5-dimethyl-7-(2,4,6-trimethylphenyl)-7H-pyrrolo[2,3-d]pyrimidin-4-yl]amino}-ethanol;
4-(1-ethyl-propyl)-2,5,6-trimethyl-7-(2,4,6-trimethylphenyl)-7H-pyrrolo[2,3-d]pyrimidine;
n-butyl-ethyl-[2,5-dimethyl-7-(2,4-dimethylphenyl)-7H-pyrrolo[2,3-d]pyrimidin4-yl]amine;
2,5-dimethyl-7-(2,4,6-trimethylphenyl)-7H-pyrrolo[2,3-d]pyrimidyl-4-yl]-(1-ethyl-propyl)amine;
butyl-[3,6-dimethyl-1-(2,4,6-trimethylphenyl)-1H-pyrazolo[3,4-b]pyridin-4-yl]-ethylamine;
[3,6-dimethyl-1-(2,4,6-trimethylphenyl)-1H-pyrazolo[3,4,b]pyridin-4-yl]-(1-methoxymethylpropyl)-amine;
4-(1-methoxymethylpropoxy)-3,6-dimethyl-1-(2,4,6-trimethylphenyl)-1H-pyrazolo[3,4-b]pyridine;
(1-ethylpropyl)-[3,5,6-trimethyl-1-(2,4,6-trimethylphenyl)-1H-pyrazolo[3,4-b]pyridin-4]-amine;
4-(1-ethylpropoxy)-2,5-dimethyl-7-(2,4,6-trimethylphenyl)-7H-pyrrolo[2,3-b]pyridine;
4-(1-ethylpropoxy)-2,5,6-trimethyl-7-(2,4,6-trimethylphenyl)-7H-pyrrolo[2,3-b]pyridine;
4-(1-ethylpropoxy)-2,5-dimethyl-7-(2,6-dimethyl-4-bromophenyl)-7H-pyrrolo[2,3-b]pyridine;
2,5,6-trimethyl-7-(1-propylbutyl)-4-(2,4,6-trimethylphenoxy)-7H-pyrrolo[2,3-d]pyrimidine;
1-(1-ethylpropyl)-6-methyl-4-(2,4,6-trimethylphenylamino)-1,3-dihydro-imidazo[4,5-c]pyridin-2-one;
9-(1-ethylpropyl)-2-methyl-6-(2,4,6-trimethylphenylamino)-7,9-dihydro-purin-8-one;
1-(1-ethylpropyl)-6-methyl-4-(2,4,6-trimethylphenoxy)-1,3-dihydro-imidazo[4,5-c]pyridin-2-one;
1-(1-ethylpropyl)-6-methyl-4-(2,4,6-trimethylphenoxy)-1H-imidazo[4,5-c]pyridine;
1-(1-ethylpropyl)-3,6-dimethyl-4-(2,4,6-trimethylphenoxy)-1,3-dihydro-imidazo[4,5-c]pyridin-2-one;
1-(1-ethylpropyl)-3,6-dimethyl-4-(2,4,6-trimethylphenylamino)-1,3-dihydro-imidazo[4,5-c]pyridin-2-one;
1-(1-ethyl-propyl)-4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,2,3,4-tetrahydro-pyrido[3,4-b]pyrazine;
1-(1-ethyl-propyl)-7-methyl-5-(2,4,6-trimethyl-phenoxy)-1,2,3,4-tetrahydro-pyrido[3,4-b]pyrazine;
1-(1-ethyl-propyl)-7-methyl-2-oxo-5-(2,4,6-trimethyl-phenoxy)-1,2,3,4-tetrahydro-[1,6]naphthyridine-3-carboxylic acid methyl ester;
1-(1-ethyl-propyl)-7-methyl-2-oxo-5-(2,4,6-trimethyl-phenoxy)-1,2,3,4-tetrahydro-[1,6]naphthyridine-3-carboxylic acid isopropyl ester;
1-(1-ethyl-propyl)-7-methyl-5-(2,4,6-trimethyl-phenoxy)-3,4-dihydro-1H-[1,6]naphthyridin-2-one;
1-(1-ethyl-propyl)-7-methyl-5-(2,4,6-trimethyl-phenoxy)-1,2,3,4-tetrahydro-[1,6]naphthyridine;
1-(1-ethyl-propyl)-7-methyl-5-(2,4,6-trimethyl-phenoxy)-1,4-dihydro-2H-3-oxa-1,6-diaza-naphthalene;
1-(1-ethyl-propyl)-4,7-dimethyl-5-(2,4,6-trimethyl-phenoxy)-1,4-dihydro-2H-3-oxa-1,6-diaza-naphthalene;
1-(1-ethyl-propyl)-3,7-dimethyl-5-(2,4,6-trimethyl-phenoxy)-3,4-dihydro-1H-3-oxa-[1,6]-naphthyridin-2-one;
1-(1-ethyl-propyl)-3,3,6-trimethyl-4-(2,4,6-trimethyl-phenoxy)-2,3-dihydro-1H-pyrrolo[3,2-c]pyridine;
7-(1-ethyl-propoxy)-5-methyl-3-(2,4,6-trimethyl-phenyl)-pyrazolo[1,5-a]pyrimidine;
[2,5-dimethyl-3-(2,4,6-trimethyl-phenyl)-pyrazolo[1,5-a]pyrimidin-7-yl]-(1-ethyl-propyl)-amine;
(1-ethyl-propyl)-[5-methyl-3-(2,4,6-trimethyl-phenyl)-pyrazolo[1,5-a]pyrimidin-7-yl]-amine;
7-(1-ethyl-propoxy)-2,5-dimethyl-3-(2,4,6-trimethyl-phenyl)-pyrazolo[1,5-a]pyrimidine;
[2,5-dimethyl-3-(2,4,6-trimethyl-phenyl)-pyrazolo[1,5-a]pyrimidin-7-yl]-ethyl-propyl-amine;
[6-bromo-5-bromomethyl-3-(2,4,6-trimethyl-phenyl)-3H-[1,2,3]triazolo[4,5-b]pyridin-7-yl]-(1-ethyl-propyl)-amine;
(1-ethyl-propyl)-[5-methyl-3-(2,4,6-trimethyl-phenyl)-3H-[1,2,3]triazolo[4,5-b]pyridin-7-yl]-amine;

[6-bromo-5-methyl-3-(2,4,6-trimethyl-phenyl)-3H-[1,2,3]triazolo[4,5-b]pyridin-7-yl]-(1-ethyl-propyl)-methyl-amine;
7-(1-ethyl-propoxy)-5-methyl-3-(2,4,6-trimethyl-phenyl)-3H-[1,2,3]triazolo[4,5-b]pyridine;
4-(1-ethyl-propoxy)-2,5-dimethyl-7-(2,4,6-trimethyl-phenyl)-5H-pyrrolo[3,2-d]pyrimidine;
(+)-2,5-dimethyl-4-(tetrahydro-furan-3-yloxy)-7-(2,4,6-trimethyl-phenyl)-5H-pyrrolo-[3,2-d]pyrimidine;
2,5-dimethyl-4-(S)-(tetrahydro-furan-3-yloxy)-7-(2,4,6-trimethyl-phenyl)-5H-pyrrolo-[3,2-d]pyrimidine;
2,5-dimethyl-4-(1-propyl-butoxy)-7-(2,4,6-trimethyl-phenyl)-5H-pyrrolo[3,2-d]pyrimidine;
4-sec-butylsulfanyl-2,5-dimethyl-7-(2,4,6-trimethyl-phenyl)-5H-pyrrolo[3,2-d]pyrimidine;
4-(butyl-ethyl-amino)-2,6-dimethyl-8-(2,4,6-trimethyl-phenyl)-5,8-dihydro-6H-pyrido[2,3-d]pyrimidin-7-one;
8-(1-ethyl-propoxy)-6-methyl-4-(2,4,6-trimethyl-phenyl)-3,4-dihydro-1H-pyrido[2,3-b]pyrazin-2-one;
8-(1-ethyl-propoxy)-6-methyl-4-(2,4,6-trimethyl-phenyl)-1,2,3,4-tetrahydro-pyrido[2,3-b]pyrazine;
4-(1-ethyl-propoxy)-2-methyl-8-(2,4,6-trimethyl-phenyl)-quinoline;
5-(1-ethyl-propoxy)-7-methyl-1-(2,4,6-trimethyl-phenyl)-1,4-dihydro-2H-3-oxa-1,8-diaza-naphthalene;
5-(1-ethyl-propoxy)-7-methyl-1-(2,4,6-trimethyl-phenyl)-1,2-dihydro-3-oxa-1,8-diaza-naphthalen-4-one;
8-(1-ethyl-propoxy)-1,6-dimethyl-4-(2,4,6-trimethyl-phenyl)-1,2,3,4-tetrahydro-pyrido[2,3-b]pyrazine;
(1-ethyl-propyl)-[2-methyl-8-(2,4,6-trimethyl-phenyl)-quinolin-4-yl]-amine;
4-(butyl-ethyl-amino)-2,6-dimethyl-8-(2,6-dimethyl-4-bromo-phenyl)-5,8-dihydro-6H-pyrido[2,3-d]pyrimidin-7-one;
4-(butyl-ethyl-amino)-2-methyl-8-(2,6-dimethyl-4-bromo-phenyl)-5,8-dihydro-6H-pyrido[2,3-d]pyrimidin-7-one;
4-(1-ethyl-propoxy)-2-methyl-8-(2,6-dimethyl-4-bromo-phenyl)-5,8-dihydro-6H-pyrido[2,3-d]pyrimidin-7-one;
(butyl-ethyl)-[2-methyl-8-(2,6-dimethyl-4-bromo-phenyl)-5,6,7,8-tetrahydro-pyrido[2,3-d]pyrimidin-4-yl]-amine;
(propyl-ethyl)-[2-methyl-8-(2,6-dimethyl-4-bromo-phenyl)-5,6,7,8-tetrahydro-pyrido[2,3-d]pyrimidin-4-yl]-amine;
(diethyl)-[2-methyl-8-(2,6-dimethyl-4-bromo-phenyl)-5,6,7,8-tetrahydro-pyrido[2,3-d]pyrimidin-4-yl]-amine;
(1-ethyl-propyl)-[2-methyl-8-(2,6-dimethyl-4-bromo-phenyl)-5,6,7,8-tetrahydro-pyrido[2,3-d]pyrimidin-4-yl]-amine;
(1-ethyl-propoxy)-2-methyl-8-(2,6-dimethyl-4-bromo-phenyl)-5,6,7,8-tetrahydro-pyrido[2,3-d]pyrimidine;
4-(butyl-ethyl-amino)-2-methyl-8-(2,4,6-trimethyl-phenyl)-5,8-dihydro-6H-pyrido[2,3-d]pyrimidin-7-one;
4-(1-ethyl-propoxy)-2-methyl-8-(2,4,6-trimethyl-phenyl)-5,8-dihydro-6H-pyrido[2,3-d]pyrimidin-7-one;
(butyl-ethyl)-[2-methyl-8-(2,4,6-trimethyl-phenyl)-5,6, 7,8-tetrahydro-pyrido[2,3-d]pyrimidin-4-yl]-amine;
(propyl-ethyl)-[2-methyl-8-(2,4,6-trimethyl-phenyl)-5,6,7,8-tetrahydro-pyrido[2,3-d]pyrimidin-4-yl]-amine;
(diethyl)-[2-methyl-8-(2,4,6-trimethyl-phenyl)-5,6,7,8-tetrahydro-pyrido[2,3-d]pyrimidin-4-yl]-amine;
(1-ethyl-propyl)-[2-methyl-8-(2,4,6-trimethyl-phenyl)-5,6,7,8-tetrahydro-pyrido[2,3-d]pyrimidin-4-yl]-amine;
(1-ethyl-propoxy)-2-methyl-8-(2,4,6-trimethyl-phenyl)-5,6,7,8-tetrahydro-pyrido[2,3-d]pyrimidine;
8-(1-ethyl-propoxy)-6-methyl-4-(2,6-dimethyl-4-bromo-phenyl)-3,4-dihydro-1H-pyrido[2,3-b]pyrazin-2-one;
8-(1-ethyl-propoxy)-6-methyl-4-(2,6-dimethyl-4-bromo-phenyl)-1,2,3,4-tetrahydro-pyrido[2,3-b]pyrazine;
4-(l -ethyl-propoxy)-2-methyl-8-(2,6-dimethyl-4-bromo-phenyl)-quinoline;
5-(1-ethyl-propoxy)-7-methyl-1-(2,6-dimethyl-4-bromo-phenyl)-1,4-dihydro-2H-3-oxa-1,8-diaza-naphthalene;
5-(1-ethyl-propoxy)-7-methyl-1-(2,6-dimethyl-4-bromo-phenyl)-1,2-dihydro-3-oxa-1,8-diaza-naphthalen-4-one;
8-(1-ethyl-propoxy)-1,6-dimethyl-4-(2,6-dimethyl-4-bromo-phenyl)-1,2,3,4-tetrahydro-pyrido[2,3-b]pyrazine;
(1-ethyl-propyl)-[2-methyl-8-(2,6-dimethyl-4-bromo-phenyl)-quinolin-4-yl]-amine;
4-(butyl-ethyl-amino)-2,6-dimethyl-8-(2,6-dimethyl-4-chloro-phenyl)-5,8-dihydro-6H-pyrido[2,3-d]pyrimidin-7-one;
8-(1-ethyl-propoxy)-6-methyl-4-(2,6-dimethyl-4-chloro-phenyl)-3,4-dihydro-1H-pyrido[2,3-b]pyrazin-2-one;
8-(1-ethyl-propoxy)-6-methyl-4-(2,6-dimethyl-4-chloro-phenyl)-1,2,3,4-tetrahydro-pyrido[2,3-b]pyrazine;
4-(1-ethyl-propoxy)-2-methyl-8-(2,6-dimethyl-4-chloro-phenyl)-quinoline;
5-(1-ethyl-propoxy)-7-methyl-1-(2,6-dimethyl-4-chloro-phenyl)-1,4-dihydro-2H-3-oxa-1,8-diaza-naphthalene;
5-(1-ethyl-propoxy)-7-methyl-1-(2,6-dimethyl-4-chloro-phenyl)-1,2-dihydro-3-oxa-1,8-diaza-naphthalen-4-one;
8-(1-ethyl-propoxy)-1,6-dimethyl-4-(2,6-dimethyl-4-chloro-phenyl)-1,2,3,4-tetrahydro-pyrido[2,3-b]pyrazine;
(1-ethyl-propyl)-[2-methyl-8-(2,6-dimethyl-4-chloro-phenyl)-quinolin-4-yl]-amine;
8-(1-hydroxymethyl-propoxy)-6-methyl-4-(2,4,6-trimethyl-phenyl)-3,4-dihydro-1H-pyrido[2,3-b]pyrazin-2-one;
8-(1-hydroxymethyl-propylamino)-6-methyl-4-(2,4,6-trimethyl-phenyl)-3,4-dihydro-1H-pyrido[2,3-b]pyrazin-2-one;
8-(1-ethyl-propylamino)-6-methyl-4-(2,4,6-trimethyl-phenyl)-3,4-dihydro-1H-pyrido[2,3-b]pyrazin-2-one;
8-diethylamino-6-methyl-4-(2,4,6-trim ethyl-phenyl)-3,4-dihydro-1H-pyrido[2,3-b]pyrazin-2-one;
8-(ethyl-propyl-amino)-6-methyl-4-(2,4,6-trimethyl-phenyl)-3,4-dihydro-1H-pyrido[2,3-b]pyrazin-2-one;
8-(butyl-ethyl-amino)-6-methyl-4-(2,4,6-trimethyl-phenyl)-3,4-dihydro-1H-pyrido[2,3-b]pyrazin-2-one;
8-(1-hydroxymethyl-propoxy)-6-methyl-4-(2,4,6-trimethyl-phenyl)-1,2,3,4-tetrahydro-pyrido[2,3-b]pyrazine;
8-(1-hydroxymethyl-propylamino)-6-methyl-4-(2,4,6-trimethyl-phenyl)-1,2,3,4-tetrahydro-pyrido[2,3-b]pyrazine;
8-(1-ethyl-propylamino)-6-methyl-4-(2,4,6-trimethyl-phenyl)-1,2,3,4-tetrahydro-pyrido[2,3-b]pyrazine;
8-diethylamino-6-methyl-4-(2,4,6-trimethyl-phenyl)-1,2,3,4-tetrahydro-pyrido[2,3-b]pyrazine;
8-(ethyl-propyl-amino)-6-methyl-4-(2,4,6-trimethyl-phenyl)-1,2,3,4-tetrahydro-pyrido[2,3-b]pyrazine;
8-(butyl-ethyl-amino)-6-methyl-4-(2,4,6-trimethyl-phenyl)-1,2,3,4-tetrahydro-pyrido[2,3-b]pyrazine;
4-(1-hydroxymethyl-propoxy)-2-methyl-8-(2,4,6-trimethyl-phenyl)-quinoline;
4-(1-hydroxymethyl-propylamino)-2-methyl-8-(2,4,6-trimethyl-phenyl)-quinoline;
4-(1-ethyl-propylamino)-2-methyl-8-(2,4,6-trimethyl-phenyl)-quinoline;
4-diethylamino-2-methyl-8-(2,4,6-trimethyl-phenyl)-quinoline;
4-(ethyl-propyl-amino)-2-methyl-8-(2,4,6-trimethyl-phenyl)-quinoline;
4-(butyl-ethyl-amino)-2-methyl-8-(2,4,6-trimethyl-phenyl)-quinoline;
5-(1-hydroxymethyl-propoxy)-7-methyl-1-(2,4,6-trimethyl-phenyl)-1,4-dihydro-2H-3-oxa-1,8-diaza-naphthalene;
5-(1-hydroxymethyl-propylamino)-7-methyl-1-(2,4,6-trimethyl-phenyl)-1,4-dihydro-2H-3-oxa-1,8-diaza-naphthalene;
5-(1-ethyl-propylamino)-7-methyl-1-(2,4,6-trimethyl-phenyl)-1,4-dihydro-2H-3-oxa-1,8-diaza-naphthalene;
5-diethylamino-5-methyl-1-(2,4,6-trimethyl-phenyl)-1,4-dihydro-2H-3-oxa-1,8-diaza-naphthalene;
5-(ethyl-propyl-amino)-7-methyl-1-(2,4,6-trimethyl-phenyl)-1,4-dihydro-2H-3-oxa-1,8-diaza-naphthalene;
8-(butyl-ethyl-amino)-6-methyl-4-(2,4,6-trimethyl-phenyl)-1,4-dihydro-2H-3-oxa-1,8-diaza-naphthalene;
4-(2,4-dichlorophenyl)-5-methyl-2-[N-(1-(methoxymethyl)-1-(naphth-2-yl)methyl)-N-propylamino]thiazole;
oxalate of 4-(2,4-dichlorophenyl)-5-methyl-2-[N-(6-methoxyisoquinol-5-yl)-N-propylamino]thiazole;
oxalate of 4-(2-chloro-4-methoxyphenyl)-5-methyl-2-[N-(6-methylisoquinol-5-yl)-N-propylamino]thiazole;
4-(2-chloro-4-methoxyphenyl)-5-methyl-2-[N-(1-methoxycarbonylmethylindol-5-yl)-N-propylamino]thiazole;
oxalate of 4-(2-chloro-4-methoxyphenyl)-5-methyl-2-[N-(6-methoxyisoquinol-5-yl)-N-propylamino]thiazole;
oxalate of 4-(2-chloro-4-methoxyphenyl)-5-methyl-2-[N-(6-chloroisoquinol-5-yl)-N-propylamino]thiazole;
oxalate of 4-(2-chloro-4-methoxyphenyl)-5-methyl-2-[N-(6-methoxyisoquinol-5-yl)-N-propylamino]thiazole;
4-(2-chloro-4-methoxyphenyl)-_5-methyl-2-[N-1-methoxynaphth-2-yl)-N-propylamino]thiazole;
oxalate of 4-(2-chloro-4-trifluoromethylphenyl)-5-methyl-2-[N-6-methoxyisoquinol-5-yl)-N-propylamino]thiazole;
chlorhydrate of 4-(20chloro-4methoxyphenyl)-5methyl-2-[N-(2-ethoxynaphth-1-yl)-N-propylamino]thiazole;
chlorhydrate of 4-(2-chloro-4-methoxyphenyl)-5-methyl-2[N-(2,3-dimethylnaphth-1-yl)-N-propylamino]thiazole;
chlorhydrate of 4-(2-chloro-4-methoxyphenyl)-5-methyl-2-[N-(6-bromo-2-methoxynaphth-1-yl)-N-propylamino]thiazole;
chlorhydrate of 4-(2-chloro-4-methoxyphenyl)-5-methyl-2-[N-(2,6-dimethylnaphth-1-yl)-N-propylamino]thiazole;
chlorhydrate of 4-(2-chloro-4-methoxyphenyl)-5-methyl-2-[N-(1-(methoxymethyl)-1-(naphth-2-yl)methyl)-N-propylamino]thiazole;
chlorhydrate of 4-(2-chloro-4-methoxyphenyl)-5-methyl-2-[N-(1-(cyclopropyl)-1-(naphth-2-yl)methyl)-N-propylamino]thiazole;
3-(2,4-dichlorophenyl)-5-methyl-7(N-propyl-N-cyclopropanemethylamino)-pyrazolo[2,3-a]pyrimidine;
3-(2,4-dichlorophenyl)-5-methyl-7-(N-allyl-N-cyclopropanemethylamino)-pyrazolo[2,3-a]pyrimidine;
2-methylthio-3-(2,4-dichlorophenyl)-5-methyl-7-(N,N-diallylamino)-pyrazolo[2,3-a]pyrimidine;
2-methylthio-3-(2,4-dichlorophenyl)-5-methyl-7-(N-butyl-N-cyclopropanemethyl-amino)pyrazolo[2,3-a]pyrimidine;
2_methylthio-3-(2,4-dichlorophenyl)-5-methyl-7-(N-propyl-N-cyclopopanemethyl-amino) pyrazolo[2,3-a]pyrimidine;
2-methyl-3-(4-chlorophenyl)-5-methyl-7-(N N-dipropylamino)-pyrazolo[2,3-a)pyrimidine;
3-[6-(dimethylamino)-3-pyridinyl-2,5-dimethyl-N,N-dipropylpyrazolo[2,3-a]pyrimidin-7-amine;
3-[6-(dimethylamino)-4-methyl-3-pyridinyl]-2,5-dimethyl-N,N-dipropyl-pyrazolo[2,3-a]pyrimidine-7-amine;
3-(2,4-dimethoxyphenyl)-2,5-dimethyl-7-(N-propyl-N-methyloxyethylamino)-pyrazolo(2,3-a)pyrimidine;
7-(N-diethylamino)-2,5-dimethyl-3-(2-methyl-4-methoxyphenyl-[1,5-a]-pyrazolopyrimidine;
7-(N-(3-cyanopropyl)-N-propylamino-2,5,dimethyl-3-(2,4-dimethylphenyl)-[1,5-a]-pyrazolopyrimidine;
[3,6-dimethyl-2-(2,4,6-trimethyl-phenoxy)-pyridin-4-yl]-(1-ethyl-propyl)-amine;
[2-(4-chloro-2,6-dimethyl-phenoxy)-3,6-dimethyl-pyridin-4-yl]-(1-ethyl-propyl)-amine;
cyclopropylmethyl-[3-(2,4-dimethyl-phenyl)-2,5-dimethyl-pyrazolo[1,5-a]pyrimidin-7-yl]-propyl-amine;
cyclopropylmethyl-[3-(2-methyl-4-chloro-phenyl)-2,5-dimethyl-pyrazolo[1,5-a]pyrimidin-7-yl]-propyl-amine;
cyclopropylmethyl-[3-(2,4-di-chloro-phenyl)-2,5-dimethyl-pyrazolo[1,5-a]pyrimidin-7-yl]-propyl-amine;
[3-(2-methyl-4-chloro-phenyl)-2,5-dimethyl-pyrazolo[1,5-a]pyrimidin-7-yl]-di-propyl-amine;
[2,5-dimethyl-3-(2,4-dimethyl-phenyl)-pyrazolo[1,5-a]pyrimidin-7-yl]-(1-ethyl-propyl)-amine;
[2,5-dimethyl-3-(2,4-dichloro-phenyl)-pyrazolo[1,5-a]pyrimidin-7-yl]-(1-ethyl-propyl)-amine; and
4-(1-Ethyl-propylamino)-6-methyl-2-(2,4,6-trimethyl-phenoxy)-nicotinic acid methyl ester.

Methods for making the CRF antagonists described above are disclosed in international patent publication WO 95/33750 incorporated by reference herein.

Particularly preferred are those compositions, methods, and kits that contain one of the following CRF antagonists:
4-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-(1-ethyl-propyl)-amine:
(3,6-dimethyl-2-(4-chloro-2,6-dimethyl-phenoxy)-pyridin-4-yl)-(1-ethyl-propyl)-amine: or
5-(1-ethyl-propoxy)-7-methyl-1-(2,6-dimethyl-4-cholorophenyl)-1-4-dihydro-2H-3-oxa-1,8-diazanaphthalene;
and one of the following atypical antipsychotics: olanazapine, clozapine, ziprasidone, or pharmaceutically acceptable salts thereof.

In the preferred kits of the present invention, the pharmaceutical composition comprising a CRF antagonist is a pharmaceutical composition comprising one of the particularly preferred CRF antagonists as defined above, and the pharmaceutical composition comprising an atypical antipsychotic is a pharmaceutical composition comprising one of the particularly preferred atypical antipsychotics as defined above.

The preferred methods of treatment of the present invention are those methods that employ a particularly preferred CRF antagonist and particularly preferred atypical antipsychotic as defined above.

Also preferred are those methods that employ a particularly preferred CRF antagonist and a particularly preferred atypical antipsychotic or a pharmaceutical composition(s) of the present invention, as defined above, for treating osteoporosis or frailty associated with aging or obesity, cardiovascular or heart related disease, in particular hypertension, tachycardia, and congestive heart failure, accelerating bone fracture repair, attenuating protein catabolic response after a major operation, reducing cachexia and protein loss due to chronic illness, accelerating wound healing, or accelerating the recovery of burn patients or of patients having undergone major surgery.

The compounds used in the present invention may have optical centers and therefore may occur in different enantiomeric configurations. The compounds used in the present invention include all enantiomers, diastereomers, and other stereoisomers of the compounds, as well as racemic and other mixtures thereof. Individual isomers can be obtained by known methods, such as optical resolution, optically selective reaction, or chromatographic separation in the preparation of the final product or its intermediate.

Preferably, the combinations of pharmaceutically active compounds of the present invention show a synergistic effect and/or show less side effects, as compared to the individual compounds, when treating a mammal, preferably a human. Thus, in treating a particular disease, at a specific dosage level, the combinations of pharmaceutically active compounds of the present invention show a better activity than the activity which could be expected when administering the individual compounds, less or less severe side effects than could be expected when administering the individual compounds, or a combination of a better activity and of less or less severe side effects than could be expected when administering the individual compounds.

The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

### DETAILED DESCRIPTION OF THE INVENTION

The compositions and combinations of this invention can be administered by oral, parenteral (e.g., intramuscular, intraperitoneal, intravenous, or subcutaneous injection, or through an implant), nasal, vaginal, rectal, sublingual, or topical routes of administration and can be formulated with pharmaceutically acceptable carriers, vehicles, or diluents to provide dosage forms appropriate for each route of administration.

Solid dosage forms for oral administration include capsules, tablets, pills, powders, granules, and the like, and for non-human mammals (cats and dogs are the presently preferred non-human mammals) the solid dosage forms can include admixtures with food and chewable forms. In such solid dosage forms, the compounds and combinations of this invention can be admixed with at least one inert pharmaceutically acceptable carrier such as sucrose, lactose, starch, or the like. Such dosage forms can also comprise, as is normal practice, additional substances other than such inert diluents, e.g., lubricating agents such as magnesium stearate. In the case of capsules, tablets, and pills, the dosage forms may also comprise buffering agents. Tablets and pills can additionally be prepared with enteric coatings. In the case of chewable forms, the dosage form may comprise flavoring agents and perfuming agents.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants (such as wetting agents), emulsifying and suspending agents, sweetening agents, flavorings, perfuming agents, and the like. Ziprazidone formulations in the form of a suspension are described in U.S. patent application Ser. No. 60/42195, filed Oct. 25, 2002 and incorporated herein by reference in its entirety. Novel injectable depot formulations of ziprasidone are described in U.S. patent application Ser. No. 60/421473, filed Oct. 25, 2002 and incorporated herein by reference in its entirety.

Preparations according to this invention for parenteral administration include sterile aqueous or non-aqueous solutions, suspensions, emulsions, and the like. Examples of non-aqueous solvents or vehicles are propylene glycol, polyethylene glycol, vegetable oils such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Compositions for rectal or vaginal administration are preferably suppositories that may contain, in addition to the active substance, excipients such as cocoa butter or a suppository wax.

Compositions for nasal or sublingual administration are also prepared with standard excipients well known in the art.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain an atypical antipsychotic in sustained or controlled release form and the CRF antagonist in an immediate release form. Alternatively, the atypical antipsychotic can be in immediate release form and the CRF antagonist can be in sustained or controlled release form.

Methods of preparing various pharmaceutical compositions with a certain amount of active ingredient are known, or will be apparent in light of this disclosure, to those skilled in this art. For examples, methods of preparing pellets are described in Remington: The Science and Practice of Pharmacy, Mack Publishing Company, Easton, Pa., 19th Edition (1995). Prolonged release pellets are prepared by either coating immediate release pellets or via matrix systems. Coating may be carried out, for example, in coating pans or in fluid bed coater-driers. Extrusion and subsequent spheronization is a long-known method for the preparation of pharmaceutical pellets (J. W. Conine et al., Drug & Cosmetic Ind. 106, 38-41 (1970)). However, other methods such as pelletization may be utilized. Particles may be agglomerated to form spherical granules or pellets, in a high-speed mixer granulator, or rotary fluid bed agglomerator. These methods are described by K. W. Olson and A. M. Mehta, Int. J. Pharm. Tech &. Prod. Mfr. 6 18-24, 1985. Pellets may be also prepared by extrusion of wet masses or melts followed by spheronisation, for example as described in C. Vervaet, L. Baert & J. P. Remon Int. J. Pharm. 116 (1995) 131-146. Excipients used are typically those with plastic qualities such as microcrystalline cellulose, but also mannitol. Small quantities of a polymeric binder are generally added. Surfactants such as sodium dodecyl sulphate may also be incorporated to give easier extrusion.

Pharmaceutical compositions according to the invention can contain 0.1%-95% of the therapeutic agents of this invention, preferably 1%-70%. In any event, the composition or formulation to be administered will contain a quantity of therapeutic agent(s) according to the invention in an amount effective to treat the condition or disease of the subject being treated.

The two active ingredients of the composition this invention can be co-administered simultaneously or sequentially in any order, or as a single pharmaceutical composition.

Pharmaceutical compositions of use in the present invention preferably comprise one or both active compound(s) in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampyules, auto-injector devices or suppositories; for oral, parenteral, intranasal, sublingual or rectal administration, or for administration by inhalation or insufflation. For preparing solid compositions such as tablets, the principal active ingredients are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof.

When referring to these preformulation compositions as homogeneous, it is meant that the active ingredients is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 2000 mg of each of the active ingredients of the present invention. Typical unit dosage forms contain from 1 to 300 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The dosage of active ingredients in the compositions and methods of this invention may be varied; however, it is necessary that the amount of the active ingredients in such compositions be such that a suitable dosage form is obtained. The selected dosage depends upon the desired therapeutic effect, on the route of administration, the particular compounds administered, the duration of the treatment, and other factors. All dosage ranges and dosage levels mentioned herein refer to each pharmaceutically active compound present in the pharmaceutical compositions and kits of the present invention, as well as those used in the methods of the present invention. Generally, dosage levels of between 0.0001 to 100 mg/kg of body weight daily are administered to humans and other animals, e.g., mammals. A preferred dosage range in humans is 0.01 to 5.0 mg/kg of body weight daily which can be administered as a single dose or divided into multiple doses. A preferred dosage range in mammals other than humans is 0.01 to 10.0 mg/kg of body weight daily which can be administered as a single dose or divided into multiple doses. A more preferred dosage range in mammals other than humans is 0.1 to 5.0 mg/kg of body weight daily which can be administered as a single dose or divided into multiple doses.

In general, the pharmaceutical compositions, methods and kits of this invention, will be administered at dosages of a therapeutically effective amount of the first and of the second therapeutic agent in single or divided doses. The term “therapeutically effective amount” as used herein refers to a sufficient amount of the compound to treat mood disorders and psychotic disorders or conditions at a reasonable benefit/risk ratio applicable to any medical treatment.

The specific therapeutically effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The following dosage amounts and other dosage amounts set forth elsewhere in this description and in the appendant claims are for an average human subject having a weight of about 65 kg to about 70 kg. The skilled practitioner will readily be able to determine the dosage amount required for a subject whose weight falls outside the 65 kg to 70 kg range, based upon the medical history of the subject. All doses set forth herein, and in the appendant claims, are daily doses.

The exact formulation, route of administration, and dosage can be chosen by the individual physician in view of the patient's condition. Dosage amount and interval can be adjusted individually to provide plasma levels of the active moiety which are sufficient to maintain therapeutic effects.

More particularly, the dosages may be as described in the patents listed herein for ziprasidone, olanzapine, clozapine, risperidone, sertindole, quetiapine, or the Physicians' Desk Reference, 57th ed., Thompson, 2003 which are expressly incorporated herein by reference. Desirably, when ziprasidone is selected as the active agent, the daily dose in the composition of the invention contains from about 5 mg to about 460 mg. More preferably, each dose of the first component contains about 20 mg to about 320 mg of the ziprasidone, and even more preferably, each dose contains from about 20 mg to about 160 mg of ziprasidone. Pediatric dosages may be less. This dosage form permits the full daily dosage to be administered in one or two oral doses, for example.

General outlines of the dosages for the atypical antipsychotics and some preferred dosages are provided herein. This list is not intended to be complete but is merely a guideline for any of the desired combinations of the present invention.

Olanzapine: from about 0.25 to about 100 mg, once/day; preferred, from about 1 to about 30 mg, once/day; and most preferably about 1 to about 25 mg once/day;
Clozapine: from about 12.5 to about 900 mg daily; preferred, from about 150 to about 450 mg daily;
Risperidone: from about 0.25 to about 16 mg daily; preferred from about 2-8 mg daily;
Sertindole: from about 0.0001 to about 1.0 mg/kg daily;
Quetiapine: from about 1.0 to about 40 mg/kg given once daily or in divided doses;
In more general terms, one would create a drug combination of the present invention by choosing a dosage of first and second component compounds according to the spirit of the above guideline.

Preferred dosage for the CRF antagonists in the composition of the invention is of about 0.01-100 mg/kg of the patient.

When administered in combination, either as a single or as separate pharmaceutical composition(s), the atypical antipsychotic and the CRF antagonist are present in a ratio which is consistent with the manifestation of the desired effect. In particular, the ratio by weight of ziprasidone to the a CRF antagonist will suitably be between 0.001 to 1 and 1000 to 1, and especially between 0.01 to 1 and 100 to 1.

The pharmaceutical combinations may be administered on a regimen of up to 6 times per day, preferably 1 to 4 times per day, especially 2 times per day, and most especially once daily.

The present invention also encompasses treatment with a combination of active ingredients which may be administered separately. Accordingly, the invention also relates to combining separate pharmaceutical compositions in kit form. Thus, in one embodiment, the kit comprises two separate pharmaceutical compositions: a corticotropin releasing factor antagonist, a prodrug thereof, or a pharmaceutically acceptable salt of said corticotropin releasing factor antagonist or said prodrug; and an atypical antipsychotic, a prodrug thereof, or a pharmaceutically acceptable salt of said atypical antipsychotic or said prodrug. The kit also comprises a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil that is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several tablets or capsules to be taken on a given day. Also, a daily dose of a corticotropin releasing factor antagonist, a prodrug thereof, or a pharmaceutically acceptable salt of said corticotropin releasing factor antagonist or said prodrug can consist of one tablet or capsule, while a daily dose of the atypical antipsychotic, prodrug thereof, or pharmaceutically acceptable salt of said atypical antipsychotic or said prodrug can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another embodiment, the present invention comprises kits comprising a pharmaceutical composition, a package, and a package insert. The pharmaceutical composition of these kits contains either a corticotropin releasing factor antagonist or an atypical antipsychotic. The kits of the present invention containing a pharmaceutical composition containing a corticotropin releasing factor antagonist differ from known kits containing a pharmaceutical composition containing a corticotropin releasing factor antagonist in that on the package and/or on the package insert of the kits it is stated that the pharmaceutical composition is to be administered together with a pharmaceutical composition containing an atypical antipsychotic. The kits of the present invention containing a pharmaceutical composition containing an atypical antipsychotic differ from known kits containing a pharmaceutical composition containing an atypical antipsychotic in that on the package and/or on the package insert of the kits it is stated that the pharmaceutical composition is to be administered together with a pharmaceutical composition containing a corticotropin releasing factor antagonist.

The term “together with” as used in the immediately preceding paragraph is intended to encompass the simultaneous administration of the two pharmaceutical compositions (e.g., a tablet containing one pharmaceutical composition is to be administered orally while the other pharmaceutical composition is administered by way of infusion, two tablets or capsules are to be swallowed together, etc.). The term “together with” is also intended to include the administration of the two pharmaceutical compositions in a specifically timed manner, i.e., one pharmaceutical composition is to be administered a certain time period after administration of the other pharmaceutical composition. The time period in which the two pharmaceutical compositions are to be administered must be sufficiently short for the corticotropin releasing factor antagonist and the atypical antipsychotic to exhibit their activity contemporaneously, preferably in a synergistic manner. The exact time period depends on the specific compounds of the pharmaceutical compositions, the application route, the kind and severeness of the disease to be treated, the kind, age, and condition of the patient to be treated, etc., and can be determined by a physician using known methods in combination with the disclosure of the present invention. Generally, the two compositions are to be administered within one day, preferably within 5 hours, more preferably within 2 hours, and even more preferably within one hour. Most preferably, the two compositions are to be administered at the same time or one immediately after the other.

Methods that may be used to determine CRF antagonist activity of the compounds employed to practice the present invention are as described in, e.g., Wynn et al., Endocrinology, 116:1653-59 (1985), and Grigoriadis et al., Peptides, 10:179-88 (1989). Methods that can be used to determine the CRF binding protein inhibiting activity of compounds employed to practice the present invention are described in Smith et al., Brain Research, 745(1,2):248-56 (1997). These methods determine the binding affinity of a test compound for a CRF receptor, which is highly related to its expected activity as a CRF antagonist.

The effectiveness of combinations of this invention, i.e., a corticotropin releasing factor antagonist and an atypical antipsychotic, may be tested for mood disorders or conditions and psychotic disorders or conditions may be demonstrated, for example, by measuring markers such as Positive or Negative Syndrome Scale (PANSS) and Scales for the Assessment of Negative Symptoms (SANS) or BPRS scores (Kay et al, 13 Schizophrenia Bulletin, 261-276; (1987)), or in various animal models such as PCP or methamphetamine induced locomotor test or the conditioned avoidance response test.

The products of the present invention have the advantage that they surprisingly provide relief from mood disorders or psychotic disorder more rapidly than would be expected from administration of either compound alone.

The invention is further illustrated by, but by no means limited to, the following examples.

### EXAMPLE 1

A pharmaceutical composition is prepared by combining ziprasidone with a CRF antagonist which is either (a) 4-(1-ethyl-propoxy)-3,6-dimethyl-2-(2,4,6-trimethylphenoxy)-pyridine, (b) (3,6-dimethyl-2-(2,4,6-trimethyl-phenoxy)-pyridin-4-yl)-(1-ethyl-propyl)-amine, (c) (3,6-dimethyl-2-(4-chloro-2,6-dimethyl-phenoxy)-pyridin-4-yl)-(1-ethyl-propyl)-amine, or (d) 5-(1-ethyl-propoxy)-7-methyl-1-(2,6-dimethyl-4-cholorophenyl)-1-4-dihydro-2 H-3-oxa-1,8-diazanaphthalene; in a pharmaceutically acceptable carrier. The composition contains respective amounts of ziprasidone and the CRF antagonist to deliver on a daily basis between about 20 mg to about 160 mg ziprasidone and between about 0.1 to 100 mg of the CRF antagonist. The composition is administered to a patient for the treatment of schizophrenia on a daily, twice daily, three times daily, or four times daily basis.

### EXAMPLE 2

Administration of ziprasidone in combination with CRF antagonists.

A prospective, open-label, randomized, flexible-dose multicenter study is carried out comparing the efficacy of IM ziprasidone with and without a CRF antagonist in the dosages of the CRF antagonist described in Example 1 in improving agitation and psychopathology in patients with psychotic disorders. Ziprasidone is given IM at a dose of 10 or 20 mg, with an additional daily dose if needed to a maximum of 40 mg.

About half of ziprasidone treated patients receive at least one dose of a CRF antagonist of Example 1 during IM therapy. Primary efficacy outcomes are mean change from baseline in Brief Psychiatric Rating Scale (BPRS), CGI-S, and CGI-Improvement (CGI-I) scores. BPRS, CGI-S, and CGI-I are rated at baseline, once every 24 hours during IM treatment, and at the end of day three.

It should be understood that the invention is not limited to the particular embodiments described herein, but that various changes and modifications may be made without departing from the spirit and scope of this novel concept as defined by the following claims.

1. A pharmaceutical compositions comprising (a) an atypical antipsychotic, a prodrug thereof or a pharmaceutically acceptable salt of the atypical antipsychotic or prodrug thereof, (b) a corticotropin releasing factor antagonist, a prodrug thereof, or pharmaceutically acceptable salt of said corticotropin releasing factor antagonist or prodrug thereof, and optionally (c) a pharmaceutically acceptable vehicle, carrier or diluent.

2. The composition of claim 1, wherein the atypical antipsychotic is a compound represented by the formula A 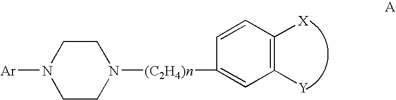
wherein Ar is benzoisothiazolyl or an oxide or dioxide thereof each optionally substituted by one fluoro, chloro, trifluoromethyl, methoxy, cyano, or nitro;
n is 1 or 2;
and X and Y together with the phenyl to which they are attached form benzothiazolyl; 2-aminobenzothiazolyl; benzoisothiazolyl; indazolyl; 2-hydroxyindazolyl; indolyl; oxindolyl optionally substituted by one to three of (C.sub.1-C.sub.3)alkyl, or one of chloro, fluoro or phenyl, said phenyl optionally substituted by one chloro or fluoro; benzoxazolyl; 2-aminobenzoxazolyl; benzoxazolonyl; 2-aminobenzoxazolinyl; benzothiazolonyl; bezoimidazolonyl; or benzotriazolyl.

3. The composition of claim 1, wherein the atypical antipsychotic is a compound represented by the formula B 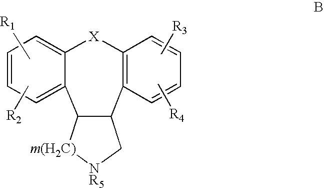
or pharmaceutically acceptable salts thereof, wherein
R₁, R₂, R₃ and R₄ each represent hydrogen, hydroxy, halogen, a C₁₋C₆ alkyl group, an alkoxy or alkylthio group in which the alkyl group contains 1-6 carbon atoms, or a trifluoromethyl group,
R₅ represents hydrogen, a C₁-C₆ alkyl group carbon atoms or an aralkyl group with 7-10 carbon atoms,
m is 1 or 2,
X represents oxygen, sulphur, the group —N(R₆)— or the group —CH₂—, and
R₆ represents hydrogen or a C₁-C₄ alkyl group.

4. The composition of claim 1, wherein the atypical antipsychotic is selected from the group consisting of olanazapine, clozapine, aripiprazole, quetiapine, amisulpride, risperidone, sertindole, asenapine, and ziprasidone.

5. The composition of claim 1 wherein said corticotropin releasing factor antagonist is a compound of the formula 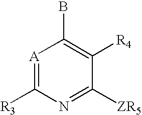
wherein
A is CR₇ or N;
B is NR₁R₂, CR₁R₂R₁₁, C(═CR₂R₁₂)R₁, NHCHR₁R₂, OCHR₁R₂, SCHR₁R₂, CHR₂OR₁₂, CHR₂SR₁₂, C(S)R₂ or C(O)R₂;
Z is NH, O, S, N (C₁-C₂ alkyl), or CR₁₃R₁₄, wherein R₁₃ and R₁₄ are each independently hydrogen, trifluoromethyl, or C₁-C₄ alkyl, or one of R₁₃ and R₁₄ may be cyano, chloro, bromo, iodo, fluoro, hydroxy, O(C₁-C₂ alkyl), amino, NH(C₁-C₂ alkyl), or CR₁₃R₁₄ may be C═O or cyclopropyl;
R₁ is C₁-C₆ alkyl which may be substituted by one or two substituents R₈ independently selected from the group consisting of hydroxy, fluoro, chloro, bromo, iodo, C₁-C₄ alkoxy, O—CO—(C₁-C₄ alkyl), O—CO—NH(C₁l-C₄ alkyl), O—CO—N(C₁-C₄ alkyl)(C₁-C₂ alkyl), NH(C₁-C₄ alkyl), N(C₁-C₂ alkyl)(C₁-C₄ alkyl), S(C₁-C₄ alkyl), N(C₁-C₄alkyl)CO(C₁-C₄ alkyl), NHCO(C₁-C₄ alkyl), COO(C₁-C₄ alkyl), CONH(C₁-C₄ alkyl), CON(C₁-C₄ alkyl)(C₁-C₂ alkyl), S(C₁-C₄ alkyl), CN, NO₂, SO(C₁-C₄ alkyl), SO₂(C₁-C₄ alkyl), and said C₁-C₆ alkyl or C₁-C₄ alkyl may contain one double or triple bond;
R₂ is C₁-C₁₂ alkyl, aryl or (C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, or benzoxazolyl; 3- to 8-membered cycloalkyl or (C₁-C₆ alkylene)cycloalkyl, wherein said cycloalkyl may contain one or two of O, S or N—R₉ wherein R₉ is hydrogen, or C₁-C₄ alkyl, wherein the above defined R₂ may be substituted independently by from one to three of chloro, fluoro, or C₁-C₄ alkyl, or one of bromo, iodo, C₁-C₆ alkoxy, O—CO—(C₁-C₆ alkyl), O—CO—N(C₁-C₄ alkyl)(C₁-C₂ alkyl), S(C₁-C₆ alkyl), CN, NO₂, SO(C₁-C₄ alkyl), or SO₂(C₁-C₄ alkyl), and wherein said C₁-C₁₂ alkyl or C₁-C₄ alkylene may contain one double or triple bond; or
NR₁R₂ or CR₁R₂R₁₁ may form a saturated 5- to 8-membered carbocyclic ring which may contain one or two double bonds or one or two of O or S;
R₃ is methyl, ethyl, fluoro, chloro, bromo, iodo, cyano, methoxy, OCF₃, methylthio, methylsulfonyl, CH₂OH or CH₂OCH₃;
R₄ is hydrogen, C₁-C₄ alkyl, fluoro, chloro, bromo, iodo, C₁-C₄ alkoxy, amino, nitro, NH(C₁-C₄ alkyl), N(C₁-C₄ alkyl)(C₁-C₂ alkyl), SOₙ(C₁-C₄ alkyl), wherein n is 0, 1 or 2, cyano, hydroxy, CO(C₁-C₄ alkyl), CHO, or COO(C₁-C₄ alkyl), wherein said C₁-C₄ alkyl may contain one or two double or triple bonds and may be substituted by one or two of hydroxy, amino, carboxy, NHCOCH₃, NH(C₁-C₂ alkyl), N(C₁-C₂ alkyl)₂, COO(C₁-C₄ alkyl), CO(C₁-C₄ alkyl), C₁-C₃ alkoxy, C₁-C₃ thioalkyl, fluoro, chloro, cyano or nitro;
R₅ is phenyl, naphthyl, thienyl, benzothienyl, pyridyl, quinolyl, pyrazinyl, pyrimidyl, furanyl, benzofuranyl, benzothiazolyl, or indolyl, wherein each one of the above groups R₅ is substituted independently by from one to three of fluoro, chloro, C₁-C₆ alkyl, or C₁-C₆ alkoxy, or one of hydroxy, iodo, bromo, formyl, cyano, nitro, trifluoromethyl, amino, NH(C₁-C₄ alkyl), N(C₁-C₆)(C₁-C₂ alkyl), COOH, COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), or SO₂(C₁-C₆ alkyl) wherein said C₁-C₄ alkyl and C₁-C₆ alkyl may be substituted by one or two of fluoro, hydroxy, amino, methylamino, dimethylamino or acetyl;
R₇ is hydrogen, C₁-C₄ alkyl, fluoro, chloro, bromo, iodo, cyano, hydroxy, O(C₁-C₄ alkyl), C(O)(C₁-C₄ alkyl), or C(O)O(C₁-C₄ alkyl), wherein the C₁-C₄ alkyl groups may be substituted with one hydroxy, chloro or bromo, or one to three fluoro;
R₁₁ is hydrogen, hydroxy, fluoro, or methoxy; and
R₁₂ is hydrogen or C₁-C₄ alkyl.

6. The composition of claim 1 wherein said corticotropin releasing factor antagonist is a compound of the formula 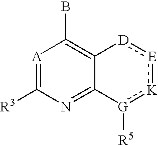
wherein the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹NHR¹, —CR²R¹⁰OR¹, —CR²R¹⁰SR¹ or —COR²;
G is nitrogen or CR⁴ and is single bonded to all atoms to which it is attached, or G is carbon and is double bonded to K;
K is nitrogen or CR⁶ when double bonded to G or E, or K is oxygen, sulfur, C═O, C═S, CR⁶R¹² or NR⁸ when single bonded to both adjacent ring atoms, or K is a two atom spacer, wherein one of the two ring atoms of the spacer is oxygen, nitrogen, sulfur, C═O, C═S, CR⁶R¹², NR⁶ or CR⁶, and the other is CR⁶R¹² or CR⁹;
D and E are each, independently, C═O, C═S, sulfur, oxygen, CR⁴R⁶ or NR⁸ when single bonded to both adjacent ring atoms, or nitrogen or CR⁴ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;
R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur atom or by NZ wherein Z is hydrogen, C₁-C₄ alkyl or benzyl, and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ² wherein Z² is hydrogen, benzyl or C₁-C₄ alkyl;
R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);
each R⁸, R⁹ and R¹² is selected, independently, from hydrogen and C₁-C₂ alkyl;
each R⁴ and R⁶ that is attached to a carbon atom is selected, independently, from hydrogen and C₁-C₆ alkyl, fluoro, chloro, bromo, iodo, hydroxy, hydroxy (C₁-C₂ alkyl), trifluoromethyl, cyano, amino, nitro, —O(C₁-C₄ alkyl), —N(C₁-C₄ alkyl)(C₁-C₂ alkyl), —CH₂SCH₃, —S(C₁-C₄ alkyl), —CO(C₁-C₄ alkyl), —C(═O)H or —C(═O)O(C₁-C₄ alkyl), wherein each of the C₁-C₂ alkyl moieties in the foregoing R⁴ and R⁶ groups may optionally contain one double or triple bond; and R⁶, when attached to a nitrogen atom, is selected from hydrogen and C₁-C₄ alkyl;
R⁵ is substituted phenyl, naphthyl, pyridyl or pyrimidyl, wherein each of the foregoing R⁵ groups is substituted with from two to four substituents R¹³, wherein up to three of said substituents may be selected, independently, from chloro, C₁-C₆ alkyl, —O(C₁C₆ alkyl) and —(C₁-C₆ alkylene)O(C₁-C₆alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —(C₀-C₁alkylene)-S—(C₁-C₂ alkyl), —(C₀-C₁alkylene)-SO—(C₁-C₂alkyl), —(C₀-C₁alkylene)-SO—(C₁-C₂alkyl) and —(C₁-C₄ alkylene)-OH, and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo (e.g., chloro, fluoro, iodo or bromo), hydroxy, methoxy, —C(═O)(C₁-C₂ alkyl), —C(═O)O(C₁-C₂ alkyl), hydroxymethyl, trifluoromethyl or formyl;
R¹⁰ is hydrogen, hydroxy, methoxy or fluoro; and
R¹¹ is hydrogen or C₁-C₄ alkyl;
with the proviso that in the ring containing D, E, K and G of formula I, there can not be two double bonds adjacent to each other.

7. The composition of claim 1 wherein said CRF antagonist is selected from the group consisting of:
4-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-(l -ethyl-propyl)-amine;
(3,6-dimethyl-2-(4-chloro-2,6-dimethyl-phenoxy)-pyridin-4-yl)-(l -ethyl-propyl)-amine;
5-(1-ethyl-propoxy)-7-methyl-1-(2,6-dimethyl-4-cholorophenyl)-1-4-dihydro-2H-3-oxa-1,8-diazanaphthalene;
butyl-[2,5-dimethyl-7-(2,4,6-trimethylphenyl)-6,7-dihydro-5H-pyrrolo[2,3-d]pyrimidin-4-yl]-ethyl-amino;
4-(butyl-ethylamino)-2,5-dimethyl-7-(2,4,6-trimethylphenyl)-5,7-dihydro-pyrrolo[2,3-d]pyrimidin-6-one;
4-(1-ethylpropoxy)-2,5-dimethyl-6-(2,4,6-trimethylphenoxy)-pyrimidine;
N-butyl-N-ethyl-2,5-dimethyl-NN-(2,4,6-trimethylphenyl)-pyrimidine-4,6-diamine;
[4-(1-ethyl-propoxy)-3,6-dimethyl-pyridin-2-yl]-(2,4,6-trimethylphenyl)-amine;
6-(ethyl-propyl-amino)-2,7-dimethyl-9-(2,4,6-trimethylphenyl)-7,9-dihydro-purin-8-one;
3-{(4-methyl-benzyl)-[3,6-dimethyl-1-(2,4,6-trimethylphenyl)-1H-pyrazolo[3,4-d]pyrimidin-4-yl]-amino}-propan-1-ol;
diethyl-[6-methyl-3-methylsulfanyl-1-(2,4,6-trichlorophenyl)-1H-pyrazolo[3,4-d]pyrimidin-4-yl]-amine;
2-{butyl-[6-methyl-3-methylsulfanyl-1-(2,4,6-trichlorophenyl)-1H-pyrazolo[3,4-d]pyrimidin-4-yl]-amino}-ethanol;
dibutyl-[6-methyl-3-methylsulfanyl-1-(2,4,6-trichlorophenyl)-1H-pyrazolo[3,4-d]pyrimidin-4-yl}-amine;
butyl-ethyl-[6-methyl-3-methylsulfanyl-1-(2,4,6-trichlorophenyl)-1H-pyrazolo[3,4-d]pyrimidin-4-yl]-amine;
butyl-ethyl-[6-methyl-3-methylsulfonyl-1-(2,4,6-trichlorophenyl)-1H-pyrazolo[3,4-d]pyrimidin-4-yl]-amine;
butyl-cyclopropylmethyl-[6-methyl-3-methylsulfanyl-1-(2,4,6-trichlorophenyl)-1H-pyrazolo[3,4-d]pyrimidin-4-yl]-amine;
di-1-propyl-[6-methyl-3-methylsulfanyl-1-(2,4,6-trichlorophenyl)-1H-pyrazolo[3,4-d]pyrimidin-4-yl]-amine;
diallyl-[6-methyl-3-methylsulfanyl-1-(2,4,6-trichlorophenyl)-1H-pyrazolo[3,4-d]pyrimidin-4-yl]-amine;
butyl-ethyl-[6-chloro-3-methylsulfanyl-1-(2,4,6-trichlorophenyl)-1H-pyrazolo[3,4-d]pyrimidin-4-yl]-amine;
butyl-ethyl-[6-methoxy-3-methylsulfanyl-1-(2,4,6-trichlorophenyl)-1H-pyrazolo[3,4-d]pyrimidin-4-yl]-amine;
propyl-ethyl-[3,6-dimethyl-1-(2,4,6-trimethylphenyl)-1H-pyrazolo[3,4-d]pyrimidin-4-yl]-amine;
4-(1-ethyl-propyl)-6-methyl-3-methylsulfanyl-1-(2,4,6-trimethylphenyl)-1H-pyrazolo[3,4-d]pyrimidine;
n-butyl-ethyl-[2,5-dimethyl-7-(2,4,6-trimethylphenyl)-7H-pyrrolo[2,3-d]pyrimidin-4-yl]amine;
di-n-propyl-[2,5-dimethyl-7-(2,4,6-trimethylphenyl)-7H-pyrrolo[2,3-d]pyrimidin-4-yl]amine;
ethyl-n-propyl-[2,5-dimethyl-7-(2,4,6-trimethylphenyl)-7H-pyrrolo[2,3-d]pyrimidin-4-yl]amine;
diethyl-2,5-dimethyl-7-(2,4,6-trimethylphenyl)-7H-pyrrolo[2,3-d]pyrimidin-4-yl]amine;
n-butyl-ethyl-[2,5,6-trimethyl-7-(2,4,6-trimethylphenyl)-7H-pyrrolo[2,3-d]pyrimidin4-yl]amine;
2-{N-n-butyl-N-[2, 5-dimethyl-7-(2,4,6-trimethylphenyl)-7H-pyrrolo[2,3-d]pyrimidin-4-yl]amino}-ethanol;
4-(1-ethyl-propyl)-2,5,6-trimethyl-7-(2,4,6-trimethylphenyl)-7H-pyrrolo[2,3-d]pyrimidine;
n-butyl-ethyl-[2,5-dimethyl-7-(2,4-dimethylphenyl)-7H-pyrrolo[2,3-d]pyrimidin-4-yl]amine;
2,5-dimethyl-7-(2,4,6-trimethylphenyl)-7H-pyrrolo[2,3-d]pyrimidyl-4-yl]-(1-ethyl-propyl)amine;
butyl-[3,6-dimethyl-1-(2,4,6-trimethylphenyl)-1H-pyrazolo[3,4-b]pyridin-4-yl]-ethylamine;
[3,6-dimethyl-1-(2,4,6-trimethylphenyl)-1H-pyrazolo[3,4,b]pyridin-4-yl]-(1-methoxymethylpropyl)-amine;
4-(1-methoxymethylpropoxy)-3,6-dimethyl-1-(2,4,6-trimethylphenyl)-1H-pyrazolo[3,4-b]pyridine;
(1-ethylpropyl)-[3,5,6-trimethyl-1-(2,4,6-trimethylphenyl)-1H-pyrazolo[3,4-b]pyridin-4-yl]-amine;
4-(1-ethylpropoxy)-2,5-dimethyl-7-(2,4,6-trimethylphenyl)-7H-pyrrolo[2,3-b]pyridine;
4-(1-ethylpropoxy)-2,5,6-trimethyl-7-(2,4,6-trimethylphenyl)-7H-pyrrolo[2,3-b]pyridine;
4-(1-ethylpropoxy)-2,5-dimethyl-7-(2,6-dimethyl-4-bromophenyl)-7H-pyrrolo[2,3-b]pyridine;
2,5,6-trimethyl-7-(1-propylbutyl)-4-(2,4,6-trimethylphenoxy)-7H-pyrrolo[2,3-d]pyrimidine;
1-(1-ethylpropyl)-6-methyl-4-(2,4,6-trimethylphenylamino)-1,3-dihydro-imidazo[4,5-c]pyridin-2-one;
9-(1-ethylpropyl)-2-methyl-6-(2,4,6-trimethylphenylamino)-7,9-dihydro-purin-8-one;
1-(1-ethylpropyl)-6-methyl-4-(2,4,6-trimethylphenoxy)-1,3-dihydro-imidazo[4,5-c]pyridin-2-one;
1-(1-ethylpropyl)-6-methyl-4-(2,4,6-trimethylphenoxy)-1H-imidazo[4,5-c]pyridine;
1-(1-ethylpropyl)-3,6-dimethyl-4-(2,4,6-trimethylphenoxy)-1,3-dihydro-imidazo[4,5-c]pyridin-2-one;
1-(1-ethylpropyl)-3,6-dimethyl-4-(2,4,6-trimethylphenylamino)-1,3-dihydro-imidazo[4,5-c]pyridin-2-one;
1-(1-ethyl-propyl)-4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,2,3,4-tetrahydro-pyrido[3,4-b]pyrazine;
1-(1-ethyl-propyl)-7-methyl-5-(2,4,6-trimethyl-phenoxy)-1,2,3,4-tetrahydro-pyrido[3,4-b]pyrazine;
1-(1-ethyl-propyl)-7-methyl-2-oxo-5-(2,4,6-trimethyl-phenoxy)-1,2,3,4-tetrahydro-[1,6]naphthyridine-3-carboxylic acid methyl ester;
1-(1-ethyl-propyl)-7-methyl-2-oxo-5-(2,4,6-trimethyl-phenoxy)-1,2,3,4-tetrahydro-[1,6]naphthyridine-3-carboxylic acid isopropyl ester;
1-(1-ethyl-propyl)-7-methyl-5-(2,4,6-trimethyl-phenoxy)-3,4-dihydro-1H-[1,6]naphthyridin-2-one;
1-(1-ethyl-propyl)-7-methyl-5-(2,4,6-trimethyl-phenoxy)-1,2,3,4-tetrahydro-[1,6]naphthyridine;
1-(1-ethyl-propyl)-7-methyl-5-(2,4,6-trimethyl-phenoxy)-1,4-dihydro-2H-3-oxa-1,6-diaza-naphthalene;
1-(1-ethyl-propyl)-4,7-dimethyl-5-(2,4,6-trimethyl-phenoxy)-1,4-dihydro-2H-3-oxa-1,6-diaza-naphthalene;
1-(1-ethyl-propyl)-3,7-dimethyl-5-(2,4,6-trimethyl-phenoxy)-3,4-dihydro-1H-3-oxa-[1,6]-naphthyridin-2-one;
1-(1-ethyl-propyl)-3,3,6-trimethyl-4-(2,4,6-trimethyl-phenoxy)-2,3-dihydro-1H-pyrrolo[3,2-c]pyridine;
7-(1-ethyl-propoxy)-5-methyl-3-(2,4,6-trimethyl-phenyl)-pyrazolo[1,5-a]pyrimidine;
[2,5-dimethyl-3-(2,4,6-trimethyl-phenyl)-pyrazolo[1,5-a]pyrimidin-7-yl]-(1-ethyl-propyl)-amine;
(1-ethyl-propyl)-[5-methyl-3-(2,4,6-trimethyl-phenyl)-pyrazolo[1,5-a]pyrimidin-7-yl]-amine;
7-(1-ethyl-propoxy)-2,5-dimethyl-3-(2,4,6-trimethyl-phenyl)-pyrazolo[1,5-a]pyrimidine;
[2,5-dimethyl-3-(2,4,6-trimethyl-phenyl)-pyrazolo[1, 5-a]pyrimidin-7-yl]-ethyl-propyl-amine;
[6-bromo-5-bromomethyl-3-(2,4,6-trimethyl-phenyl)-3H-[1,2,3]triazolo[4,5-b]pyridin-7-yl]-(1-ethyl-propyl)-amine;
(1-ethyl-propyl)-[5-methyl-3-(2,4,6-trimethyl-phenyl)-3H-[1,2,3]triazolo[4,5-b]pyridin-7-yl]-amine;
[6-bromo-5-methyl-3-(2,4,6-trimethyl-phenyl)-3H-[1 2,3]triazolo[4,5-b]pyridin-7-yl]-(1-ethyl-propyl)-methyl-amine;
7-(1-ethyl-propoxy)-5-methyl-3-(2,4,6-trim ethyl-phenyl)-3H-[1,2,3]triazolo[4,5-b]pyridine;
4-(1-ethyl-propoxy)-2,5-dimethyl-7-(2,4,6-trimethyl-phenyl)-5H-pyrrolo[3,2-d]pyrimidine;
(+)-2,5-dimethyl-4-(tetrahydro-furan-3-yloxy)-7-(2,4,6-trimethyl-phenyl)-5H-pyrrolo-[3,2-d]pyrimidine;
2,5-dimethyl-4-(S)-(tetrahydro-furan-3-yloxy)-7-(2,4,6-trimethyl-phenyl)-5H-pyrrolo-[3,2-d]pyrimidine;
2,5-dimethyl-4-(1-propyl-butoxy)-7-(2,4,6-trimethyl-phenyl)-5H-pyrrolo[3,2-d]pyrimidine;
4-sec-butylsulfanyl-2,5-dimethyl-7-(2,4,6-trimethyl-phenyl)-5H-pyrrolo[3,2-d]pyrimidine;
4-(butyl-ethyl-amino)-2,6-dimethyl-8-(2,4,6-trimethyl-phenyl)-5,8-dihydro-6H-pyrido[2,3-d]pyrimidin-7-one;
8-( -ethyl-propoxy)-6-methyl-4-(2,4,6-trimethyl-phenyl)-3,4-dihydro-1H-pyrido[2,3-b]pyrazin-2-one;
8-( -ethyl-propoxy)-6-methyl-4-(2,4,6-trimethyl-phenyl)-1,2,3,4-tetrahydro-pyrido[2,3-b]pyrazine;
4-(1-ethyl-propoxy)-2-methyl-8-(2,4,6-trimethyl-phenyl)-quinoline;
5-(1-ethyl-propoxy)-7-methyl-1-(2,4,6-trimethyl-phenyl)-1,4-dihydro-2H-3-oxa-1,8-diaza-naphthalene;
5-(l -ethyl-propoxy)-7-methyl-1-(2,4,6-trimethyl-phenyl)-1,2-dihydro-3-oxa-1,8-diaza-naphthalen-4-one;
8-(1-ethyl-propoxy)-1,6-dimethyl-4-(2,4,6-trimethyl-phenyl)-1,2,3,4-tetrahydro-pyrido[2,3-b]pyrazine;
(1-ethyl-propyl)-[2-methyl-8-(2,4,6-trimethyl-phenyl)-quinolin-4-yl]-amine;
4-(butyl-ethyl-amino)-2,6-dimethyl-8-(2,6-dimethyl-4-bromo-phenyl)-5,8-dihydro-6H-pyrido[2,3-d]pyrimidin-7-one;
4-(butyl-ethyl-amino)-2-methyl-8-(2,6-dimethyl-4-bromo-phenyl)-5,8-dihydro-6H-pyrido[2,3-d]pyrimidin-7-one;
4-(1-ethyl-propoxy)-2-methyl-8-(2,6-dimethyl-4-bromo-phenyl)-5,8-dihydro-6H-pyrido[2,3-d]pyrimidin-7-one;
(butyl-ethyl)-[2-methyl-8-(2,6-dimethyl-4-bromo-phenyl)-5,6,7,8-tetrahydro-pyrido[2,3-d]pyrimidin-4-yl]-amine;
(propyl-ethyl)-[2-methyl-8-(2,6-dimethyl-4-bromo-phenyl)-5,6,7,8-tetrahydro-pyrido[2,3-d]pyrimidin-4-yl]-amine;
(diethyl)-[2-methyl-8-(2,6-dimethyl-4-bromo-phenyl)-5,6,7,8-tetrahydro-pyrido[2,3-d]pyrimidin4-yl]-amine;
(1-ethyl-propyl)-[2-methyl-8-(2,6-dimethyl-4-bromo-phenyl)-5,6,7,8-tetrahydro-pyrido[2,3-d]pyrimidin4-yl]-amine;
(1-ethyl-propoxy)-2-methyl-8-(2,6-dimethyl-4-bromo-phenyl)-5,6,7,8-tetrahydro-pyrido[2,3-d]pyrimidine;
4-(butyl-ethyl-amino)-2-methyl-8-(2,4,6-trimethyl-phenyl)-5,8-dihydro-6H-pyrido[2,3-d]pyrimidin-7-one;
4-( -ethyl-propoxy)-2-methyl-8-(2,4,6-trimethyl-phenyl)-5,8-dihydro-6H-pyrido[2,3-d]pyrimidin-7-one;
(butyl-ethyl)-[2-methyl-8-(2,4,6-trimethyl-phenyl)-5,6,7,8-tetrahydro-pyrido[2,3-d]pyrimidin-4-yl]-amine;
(propyl-ethyl)-[2-methyl-8-(2,4,6-trimethyl-phenyl)-5,6,7,8-tetrahydro-pyrido[2,3-d]pyrimidin-4-yl]-amine;
(diethyl)-[2-methyl-8-(2,4,6-trimethyl-phenyl)-5,6,7,8-tetrahydro-pyrido[2,3-d]pyrimidin-4-yl]-amine;
(1-ethyl-propyl)-[2-methyl-8-(2,4,6-trimethyl-phenyl)-5,6,7,8-tetrahydro-pyrido[2,3-d]pyrimidin-4-yl]-amine;
(1-ethyl-propoxy)-2-methyl-8-(2,4,6-trimethyl-phenyl)-5,6,7,8-tetrahydro-pyrido[2,3-d]pyrimidine;
8-(1-ethyl-propoxy)-6-methyl-4-(2,6-dimethyl-4-bromo-phenyl)-3,4-dihydro-1H-pyrido[2,3-b]pyrazin-2-one;
8-(1-ethyl-propoxy)-6-methyl-4-(2,6-dimethyl-4-bromo-phenyl)-1,2,3,4-tetrahydro-pyrido[2,3-b]pyrazine;
4-(1-ethyl-propoxy)-2-methyl-8-(2,6-dimethyl-4-bromo-phenyl)-quinoline;
5-(1-ethyl-propoxy)-7-methyl-1-(2,6-dimethyl-4-bromo-phenyl)-1,4-dihydro-2H-3-oxa-1,8-diaza-naphthalene;
5-(1-ethyl-propoxy)-7-methyl-1-(2,6-dimethyl-4-bromo-phenyl)-1,2-dihydro-3-oxa-1,8-diaza-naphthalen-4-one;
8-(1-ethyl-propoxy)-1,6-dimethyl-4-(2,6-dimethyl-4-bromo-phenyl)-1,2,3,4-tetrahydro-pyrido[2,3-b]pyrazine;
(1-ethyl-propyl)-[2-m ethyl-8-(2,6-dimethyl-4-bromo-phenyl)-quinolin-4-yl]-amine ;
4-(butyl-ethyl-amino)-2,6-dimethyl-8-(2,6-dimethyl-4-chloro-phenyl)-5,8-dihydro-6H-pyrido[2,3-d]pyrimidin-7-one;
8-(1-ethyl-propoxy)-6-methyl-4-(2,6-dimethyl-4-chloro-phenyl)-3,4-dihydro-1H-pyrido[2,3-b]pyrazin-2-one;
8-(1-ethyl-propoxy)-6-methyl-4-(2,6-dimethyl-4-chloro-phenyl)-1,2,3,4-tetrahydro-pyrido[2,3-b]pyrazine;
4-(1-ethyl-propoxy)-2-methyl-8-(2,6-dimethyl-4-chloro-phenyl)-quinoline;
5-(1-ethyl-propoxy)-7-methyl-1-(2,6-dimethyl-4-chloro-phenyl)-1,4-dihydro-2H-3-oxa-1,8-diaza-naphthalene;
5-(1-ethyl-propoxy)-7-methyl-1-(2,6-dimethyl-4-chloro-phenyl)-1,2-dihydro-3-oxa-1,8-diaza-naphthalen-4-one;
8-(1-ethyl-propoxy)-1,6-dimethyl-4-(2,6-dimethyl-4-chloro-phenyl)-1,2,3,4-tetrahydro-pyrido[2,3-b]pyrazine;
(1-ethyl-propyl)-[2-methyl-8-(2,6-dimethyl-4-chloro-phenyl)-quinolin-4-yl]-amine;
8-(1-hydroxymethyl-propoxy)-6-methyl-4-(2,4,6-trimethyl-phenyl)-3,4-dihydro-1H-pyrido[2,3-b]pyrazin-2-one;
8-(1-hydroxymethyl-propylamino)-6-methyl-4-(2,4,6-trimethyl-phenyl)-3,4-dihydro-1H-pyrido[2,3-b]pyrazin-2-one;
8-(1-ethyl-propylamino)-6-methyl-4-(2,4,6-trimethyl-phenyl)-3,4-dihydro-1H-pyrido[2,3-b]pyrazin-2-one;
8-diethylamino-6-methyl-4-(2,4,6-trimethyl-phenyl)-3,4-dihydro-1H-pyrido[2,3-b]pyrazin-2-one;
8-(ethyl-propyl-amino)-6-methyl-4-(2,4,6-trimethyl-phenyl)-3,4-dihydro-1H-pyrido[2,3-b]pyrazin-2-one
8-(butyl-ethyl-amino)-6-methyl-4-(2,4,6-trimethyl-phenyl)-3,4-dihydro-1H-pyrido[2,3-b]pyrazin-2-one;
8-(1-hydroxymethyl-propoxy)-6-methyl-4-(2,4,6-trimethyl-phenyl)-1,2,3,4-tetrahydro-pyrido[2,3-b]pyrazine;
8-(1-hydroxymethyl-propylamino)-6-methyl-4-(2,4,6-trimethyl-phenyl)-1,2,3,4-tetrahydro-pyrido[2,3-b]pyrazine;
8-(1-ethyl-propylamino)-6-methyl-4-(2,4,6-trimethyl-phenyl)-1,2,3,4-tetrahydro-pyrido[2,3-b]pyrazine;
8-diethylamino-6-methyl-4-(2,4,6-trimethyl-phenyl)-1,2,3,4-tetrahydro-pyrido[2,3-b]pyrazine;
8-(ethyl-propyl-amino)-6-methyl-4-(2,4,6-trimethyl-phenyl)-1,2,3,4-tetrahydro-pyrido[2,3-b]pyrazine;
8-(butyl-ethyl-amino)-6-methyl-4-(2,4,6-trimethyl-phenyl)-1,2,3,4-tetrahydro-pyrido[2,3-b]pyrazine;
4-(1-hydroxymethyl-propoxy)-2-methyl-8-(2,4,6-trimethyl-phenyl)-quinoline;
4-(1-hydroxymethyl-propylamino)-2-methyl-8-(2,4,6-trimethyl-phenyl)-quinoline;
4-(1-ethyl-propylamino)-2-methyl-8-(2,4,6-trimethyl-phenyl)-quinoline;
4-diethylamino-2-methyl-8-(2,4,6-trimethyl-phenyl)-quinoline;
4-(ethyl-propyl-amino)-2-methyl-8-(2,4,6-trimethyl-phenyl)-quinoline;
4-(butyl-ethyl-amino)-2-methyl-8-(2,4,6-trimethyl-phenyl)-quinoline;
5-(1-hydroxymethyl-propoxy)-7-methyl-1-(2,4,6-trim ethyl-phenyl)-1,4-dihydro-2H-3-oxa-1,8-diaza-naphthalene;
5-(1-hydroxymethyl-propylamino)-7-methyl-1-(2,4,6-trimethyl-phenyl)-1,4-dihydro-2H-3-oxa-1,8-diaza-naphthalene;
5-(1-ethyl-propylamino)-7-methyl-1-(2,4,6-trimethyl-phenyl)-1,4-dihydro-2H-3-oxa-1,8-diaza-naphthalene;
5-diethylamino-5-methyl-i -(2,4,6-trimethyl-phenyl)-1,4-dihydro-2H-3-oxa-1,8-diaza-naphthalene;
5-(ethyl-propyl-amino)-7-methyl-1-(2,4,6-trimethyl-phenyl)-1,4-dihydro-2H-3-oxa-1,8-diaza-naphthalene;
8-(butyl-ethyl-amino)-6-methyl-4-(2,4,6-trimethyl-phenyl)-1,4-dihydro-2H-3-oxa-1,8-diaza-naphthalene;
4-(2,4-dichlorophenyl)-5-methyl-2-[N-(1-(methoxymethyl)-1-(naphth-2-yl)methyl)-N-propylamino]thiazole;
oxalate of 4-(2,4-dichlorophenyl)-5-methyl-2-[N-(6-methoxyisoquinol-5-yl)-N-propylamino]thiazole;
oxalate of 4-(2-chloro-4-methoxyphenyl)-5-methyl-2-[N-(6-methylisoquinol-5-yl)-N-propylamino]thiazole;
4-(2-chloro-4-methoxyphenyl)-5-methyl-2-[N-(1-methoxycarbonylmethylindol-5-yl)-N-propylamino]thiazole;
oxalate of 4-(2-chloro-4-methoxyphenyl)-5-methyl-2-[N-(6-methoxyisoquinol-5-yl)-N-propylamino]thiazole;
oxalate of 4-(2-chloro-4-methoxyphenyl)-5-methyl-2-[N-(6-chloroisoquinol-5-yl)-N-propylamino]thiazole;
oxalate of 4-(2-chloro-4-methoxyphenyl)-5-methyl-2-[N-(6-methoxyisoquinol-5-yl)-N-propylamino]thiazole;
4-(2-chloro-4-methoxyphenyl)-5-methyl-2-[N-1-methoxynaphth-2-yl)-N-propylamino]thiazole;
oxalate of 4-(2-chloro-4-trifluoromethylphenyl)-5-methyl-2-[N-6-methoxyisoquinol-5-yl)-N-propylamino]thiazole;
chlorhydrate of 4-(2-chloro-4-methoxyphenyl)-5-methyl-2-[N-(2-ethoxynaphth-1-yl)-N-propylamino]thiazole;
chlorhydrate of 4-(2-chloro-4-methoxyphenyl)-5-methyl-2[N-(2,3-dimethylnaphth-1-yl)-N-propylamino]thiazole;
chlorhydrate de 4-(2-chloro-4-methoxyphenyl)-5-methyl-2-[N-(6-bromo-2-methoxynaphth-1-yl)-N-propylamino]thiazole;
chlorhydrate of 4-(2-chloro-4-methoxyphenyl)-5-methyl-2-[N-(2,6-dimethylnaphth-1-yl)-N-propylamino]thiazole;
chlorhydrate of 4-(2-chloro-4-methoxyphenyl)-5-methyl-2-[N-(1-(methoxymethyl)-1-(naphth-2-yl)methyl)-N-propylamino]thiazole;
chlorhydrate of 4-(2-chloro-4-methoxyphenyl)-5-methyl-2-[N-(1-(cyclopropyl)-1-(naphth-2-yl)methyl)-N-propylamino]thiazole;
3-(2,4-dichlorophenyl)-5-methyl-7(N-propyl-N-cyclopropanemethylamino)-pyrazolo[2,3-a]pyrimidine;
3-(2,4-dichlorophenyl)-5-methyl-7-(N-allyl-N-cyclopropanemethylamino)-pyrazolo[2,3-a]pyrimidine;
2-methylthio-3-(2,4-dichlorophenyl)-5-methyl-7-(N,N-diallylamino)-pyrazolo[2,3-a]pyrimidine;
2-methylthio-3-(2,4-dichlorophenyl)-5-methyl-7-(N-butyl-N-cyclopropanemethyl-amino)pyrazolo[2,3-a]pyrimidine; 2_methylthio-3-(2,4-dichlorophenyl)-5-methyl-7-(N-propyl-N-cyclopopanemethyl-amino) pyrazolo[2,3-a]pyrimidine;
2-methyl-3-(4-chlorophenyl)-5-methyl-7-(N,N-dipropylamino)-pyrazolo[2,3-a)pyrimidine;
3-[6-(dimethylamino)-3-pyridinyl-2,5-dimethyl-N,N-dipropylpyrazolo[2,3-a]pyrimidin-7-amine;
3-[6-(dimethylamino)-4-methyl-3-pyridinyl]-2,5-dimethyl-N,N-dipropyl-pyrazolo[2,3-a]pyrimidine-7-amine;
3-(2,4-dimethoxyphenyl)-2,5-dimethyl-7-(N-propyl-N-methyloxyethylamino)-pyrazolo(2,3-a)pyrimidine;
7-(N-diethylamino)-2,5-dimethyl-3-(2-methyl-4-methoxyphenyl-[1,5-a]-pyrazolopyrimidine;
7-(N-(3-cyanopropyl)-N-propylamino-2,5,dimethyl-3-(2,4-dimethylphenyl)-[1,5-a]-pyrazolopyrimidine;
[3,6-dimethyl-2-(2,4,6-trimethyl-phenoxy)-pyridin-4-yl]-(1-ethyl-propyl)-amine;
[2-(4-chloro-2,6-dimethyl-phenoxy)-3,6-dimethyl-pyridin-4-yl]-(1-ethyl-propyl)-amine;
cyclopropylmethyl-[3-(2,4-dimethyl-phenyl)-2,5-dimethyl-pyrazolo[1,5-a]pyrimidin-7-yl]-propyl-amine;
cyclopropylmethyl-[3-(2-methyl-4-chloro-phenyl)-2,5-dimethyl-pyrazolo[1,5-a]pyrimidin-7-yl]-propyl-amine;
cyclopropylmethyl-[3-(2,4-di-chloro-phenyl)-2,5-dimethyl-pyrazolo[1,5-a]pyrimidin-7-yl]-propyl-amine;
[3-(2-methyl-4-chloro-phenyl)-2,5-dimethyl-pyrazolo[1,5-a]pyrimidin-7-yl]-di-propyl-amine;
[2,5-dimethyl-3-(2,4-dimethyl-phenyl)-pyrazolo[1,5-a]pyrimidin-7-yl]-(1-ethyl-propyl)-amine;
[2,5-dimethyl-3-(2,4-dichloro-phenyl)-pyrazolo[1,5-a]pyrimidin-7-yl]-(1-ethyl-propyl)-amine; and
4-(1-Ethyl-propylamino)-6-methyl-2-(2,4,6-trimethyl-phenoxy)-nicotinic acid methyl ester.

8. The composition of claim 7, wherein the corticotropin releasing factor antagonist is selected from the group consisting of:
4-(1-ethyl-propoxy)-3,6-dimethyl-2-(2,4,6-trimethyl-phenoxy)-pyridine;
(3,6-dimethyl-2-(2,4,6-trimethyl-phenoxy)-pyridin-4-yl)-(1-ethyl-propyl)-amine;
(3,6-dimethyl-2-(4-chloro-2,6-dimethyl-phenoxy)-pyridin-4-yl)-(1-ethyl-propyl)-amine;
and 5-(1-ethyl-propoxy)-7-m ethyl-i -(2,6-dimethyl-4-cholorophenyl)-1-4-dihydro-2H-3-oxa-1,8-diazanaphthalene.

9. The composition of claim 8, wherein the atypical antipsychotic is selected from the group consisting of ziprasidone and asenapine.

10. A method for treating mood disorders or conditions, psychotic disorders or conditions, or a combination thereof, in a mammal, the method comprising administering to a mammal in need of such treatment (a) an atypical antipsychotic, a prodrug thereof or a pharmaceutically acceptable salt of the atypical antipsychotic or prodrug thereof, and (b) a corticotropin releasing factor antagonist, a prodrug thereof, or pharmaceutically acceptable salt of said corticotropin releasing factor antagonist or prodrug thereof, wherein (a) and (b) are each optionally and independently administered together with a pharmaceutically acceptable vehicle, carrier or diluent.

11. The method of claim 10, wherein the corticotropin releasing factor antagonist is selected from the group consisting of:
4-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-(1-ethyl-propyl)-amine;
(3,6-dimethyl-2-(4-chloro-2,6-dimethyl-phenoxy)-pyridin-4-yl)-(1-ethyl-propyl)-amine;
and 5-(1-ethyl-propoxy)-7-methyl-1-(2,6-dimethyl-4-cholorophenyl)-1-4-dihydro-2H-3-oxa-1,8-diazanaphthalene.

12. The method of claim 11, wherein the atypical antipsychotic is selected from the group consisting of ziprasidone and asenapine.

13. The method of claim 10 wherein the atypical antipsychotic and the corticotropin releasing factor antagonist are administered simultaneously or in a specifically timed manner.

14. A method for treating a depressive symptom associated with mood disorders or conditions, psychotic disorders or conditions, or a combination thereof, in a mammal, the method comprising administering to a mammal in need of such treatment (a) an atypical antipsychotic, a prodrug thereof or a pharmaceutically acceptable salt of the atypical antipsychotic or prodrug thereof, and (b) a corticotropin releasing factor antagonist, a prodrug thereof, or pharmaceutically acceptable salt of said corticotropin releasing factor antagonist or prodrug thereof, wherein (a) and (b) are each optionally and independently administered together with a pharmaceutically acceptable vehicle, carrier or diluent, wherein the symptom is selected from the group consisting of depressed mood, irritability, sad effect, and circadian rhythm alteration.

15. A kit comprising a pharmaceutical composition comprising a corticotropin releasing factor antagonist, a prodrug thereof, or pharmaceutically acceptable salt of said corticotropin releasing factor antagonist or prodrug thereof, a package containing the composition, and a package insert that is optionally integral with the package, wherein it is stated on the package insert that the pharmaceutical composition is to be administered to the mammal simultaneously or in a specifically timed manner with a pharmaceutical composition containing an atypical antipsychotic, a prodrug thereof, or pharmaceutically acceptable salt of said atypical antipsychotic or prodrug thereof.
